(19) United States
(12) Patent Application Publication
(10) Pub. No.: US 2004/0076546 A1
(43) Pub. Date: Apr. 22, 2004

(54) Automated kinetic solubility assay apparatus and method

(76) Inventor: Brian D. Bissett, Waterford, CT (US)

Correspondence Address: PFIZER INC., PATENT DEPARTMENT, MS8260-1611, EASTERN POINT ROAD, GROTON, CT, 06340, US

(21) Appl. No.: 10/686,464

(22) Filed: Oct. 14, 2003

Related U.S. Application Data
(60) Provisional application No. 60/419,773, filed on Oct. 18, 2002.

(51) Int. Cl. G01N 33/00

(52) U.S. Cl. 422/68.1; 422/100

(57) ABSTRACT

An automated kinetic solubility assay apparatus that uses turbidimetric means to assess the kinetic solubility of a succession of testable mixtures is disclosed. A large number of sample containers (one test compound per container), each having a pierceable septum, may be arranged for sequential testing, and the apparatus automatically (i.e., without operator intervention) tests the compounds one at a time to determine the kinetic solubility of each compound (e.g., a drug candidate being preliminarily screened) in a test fluid of interest (e.g., a liquid that mimics gastric juices). A cuvette in which the test mixture is exposed to the testing light and having a pierceable septum, a septum-piercing needle that is used to transfer the compound from its container to the cuvette, and a cleaning solution that may be used to clean the cuvette, all of which can be used in the automated apparatus, are also disclosed.

This application claims priority, under 35 U.S.C. § 119(e), from U.S. Provisional Application Ser. No. 60/419,773, filed Oct. 18, 2002.

### COMPUTER PROGRAM LISTING APPENDIX

A source code listing of a computer program (algorithm) is part of this application and disclosure and is hereby incorporated herein in its entirety for all purposes. The source code listing is submitted as a computer program listing appendix on a compact disc in accordance with 37 C.F.R. § 1.96(c), as required by the United States Patent and Trademark Office, and that appendix is and will be incorporated herein in its entirety for all purposes.

### BACKGROUND OF THE INVENTION AND OTHER INFORMATION

This invention concerns an automated kinetic solubility assay apparatus and, more specifically, an automated kinetic solubility assay apparatus that uses turbidimetric means to assess the kinetic solubility of a testable mixture. Additionally, the invention concerns a cuvette, a needle, and cleaning solutions that may be used in the automated kinetic solubility assay apparatus.

Measuring turbidity (e.g., by infrared red spectroscopy) of a mixture (e.g., solution or suspension) to indicate the kinetic solubility of a compound is discussed in various documents. See, e.g., PCT Publication WO 01/55698; Lipinski et al., “Experimental and computational approaches to estimate solubility and permeability in drug discovery and development settings,” Advanced Drug Delivery Reviews, volume 23, pages 3-25 (1997); Quarterman et al., “Improving the Odds—High Throughput Techniques in New Drug Selection,” European Pharmaceutical Review, volume 18, number 4, pages 27-32 (1998); and Bevan et al., “A High-Throughput Screening Method for the Determination of Aqueous Drug Solubility Using Laser Nephelometry in Microtiter Plates,” Analytical Chemistry, volume 72, number 8, pages 1781-1787 (2000). (All of the documents discussed or otherwise referenced herein are incorporated herein in their entireties for all purposes.)

Combinatorial chemistry synthesis has revolutionized modern drug discovery by enabling rapid production of large numbers of compounds as possible drug candidates. Workers in the field have had to develop high-throughput screening methods to quickly screen the large number of compounds that are synthesized. Assays that identify compounds having desirable physical and chemical properties early in the screening process are invaluable in identifying compounds likely to be more promising as drugs, thereby reducing the resources expended on compounds likely to be less promising. Because of the wide acceptance and convenience of orally administered drugs, drug compounds having superior aqueous solubility are often more valuable and compounds having inferior aqueous solubility are often less valuable. Therefore, assays that accurately and quickly indicate the aqueous solubility of drug candidates are highly desirable.

Unfortunately, determining the solubility of a compound can be time- and labor-intensive. Thermodynamic (or “equilibrium”) solubility is typically measured by mixing the compounds and solvents of interest and agitating for extended periods of time, usually at least 24 hours, to produce saturated solutions (i.e., the “shake-flask” method). These saturated solutions may then be filtered and analyzed by suitable analytical methods (e.g., high-performance liquid chromatography) to determine the concentrations of the dissolved compounds. Unfortunately, this method is impractical to use as a high-throughput screen (assay).

Workers in the field have suggested that determining kinetic solubility instead of thermodynamic solubility might be adequate for a high-throughput solubility screen (see WO 01/55698, Lipinski, Quarterman, and Bevan, above). Kinetic solubility is time-dependent whereas thermodynamic solubility is not. Thus, as increasing amounts of time are allowed for contact of solute and solvent before the raw solubility data are taken, the values determined for kinetic solubility approach the value for thermodynamic (true) solubility.

Kinetic solubility may be indicated by the point at which a compound precipitates out of solution because of further addition of the compound, thereby forming a suspension, or the point at which a suspension of the compound becomes a solution because of further addition of the solvent (in each case allowing less than infinite time for equilibration to occur). For example, a known quantity of a compound is added to a known quantity of solvent with agitation. After a short period of time, the mixture is examined for the presence of suspended particles. If none are present, another known quantity of the compound is added and the mixture is again examined for the presence of suspended particles. This procedure is repeated until suspended particles are detected, at which point kinetic solubility is assessed. An alternative method starts with a saturated solution also containing particles of the compound in suspension (in other words, a mixture). A known quantity of solvent is added to dilute the mixture. The mixture is then examined for the presence of suspended particles. If they are present, another known quantity of solvent is added and the mixture is again examined for the presence of suspended particles. This procedure is repeated until suspended particles are no longer detected, at which point a kinetic solubility is assessed. Turbidimetric means may be employed to detect the presence or absence of particles in the mixture. Instruments that determine the concentration or size of particles in a suspension by means of transmitted or reflected light are more generally known as nephelometers.

The turbidity of a liquid containing particulate matter is measured under specified conditions and is based on the amount of energy (e.g., light) scattered by suspended particles when energy passes through the liquid. The degree of turbidity cannot be directly equated to the concentration of suspended particles because the properties of some particles (e.g., color) can affect the scattering of the type of energy (e.g., white particles reflect more light than dark-colored particles and many small particles may together reflect more light than a large particle of equal mass or surface area). Turbidity is commonly measured in Nephelometric Turbidity Units (“NTU”), but may also be measured in Jackson Turbidity Units (“JTU”), Nephelos (“NEPH”; 6.7 NEPHs equals 1 NTU), or European Brewery Convention units (“EBC”; 1 NTU equals 0.245 EBCs).

Attempts to provide automated high-throughput turbidimetric screening methods (e.g., using infrared spectroscopy analysis) have been made (see the documents cited above), but none of those methods has proved entirely satisfactory. Some of the drawbacks of and problems encountered with those methods are poor reproducibility, and/or the need to use large amounts of solvents, and/or excessive man-hours attending to systems that are not fully automated, and/or the lack of industry-wide standardized methodology. Poor reproducibility is chief among these and is caused in part by the variability of light scattering according to particle size and particle color, sedimentation or crystallization of substances in the sample, and adhesion of substances to the walls of vessels used during turbidimetric analysis. Such adhesion is particularly a problem with substances that are gummy or tar-like. Unfortunately, the passage of time has increased the proportion of new compounds to be screened that have lower aqueous solubility (e.g., are gummy or tar-like) because the families or libraries of the more soluble compounds have tended (for obvious reasons) to be screened sooner. Separate and apart from these problems and drawbacks, there is a small (but possibly increasing) proportion of compounds to be screened that may be injurious to an operator (e.g., because of inhalation or contact with the skin), and known apparatus and methods may not satisfactorily protect operators from contact.

The assignee of the present application several years ago used a turbidimetric screening method employing a Hach 2100 turbidimeter. A sample rack that could hold up to ninety-six sample vials, each of which had to be manually opened (i.e., uncapped) by an operator (and after the assay, manually closed, i.e., capped, by an operator), were placed on a tray proximate the turbidimeter. A robotic arm moved a needle (a fused silica capillary) to a position over and then down into each of the open vials, and a sample (a dimethyl sulfoxide solution containing the substance whose turbidity in a test fluid was to be determined) was withdrawn up into the passageway within the needle. The test fluid (e.g., a pH 7 chloride-free buffered solution) was placed in a mixing chamber located outside of the turbidimeter and containing a magnetic mixing bar, the needle containing the test substance in dimethyl sulfoxide was moved to the mixing chamber, which was open at the top, and a small aliquot of the test substance (in dimethyl sulfoxide) was then dispensed from the needle into mixing chamber. The mixture was pumped from the mixing chamber into the testing chamber (a rectangular lateral cross-section 2.5 milliliter volume stock cuvette from Starna Cells, Inc., Atascadero, Calif.), and the turbidimetric reading was taken by the turbidimeter. To add more of the substance to the mixture (to increase the concentration of substance), the mixture was pumped out of the cuvette back into the mixing chamber and an additional aliquot of substance was dispensed by the needle into the mixing chamber. After sufficient mixing (by the magnetic stir bar), the mixture with the additional aliquot of substance was pumped back into the cuvette for the next turbidimetric reading. After the reading on the final mixture was taken (i.e., after the maximum allowable number of additions of test substance had been made or the measured turbidity exceeded a specified value), the testing chamber was emptied and rinsed with methanol. The methanol rinse was sufficient to clean the testing chamber because most (if not all) of any deposition of the substance being tested occurred in the separate mixing chamber. After the last addition of the substance being tested was made by the needle into the mixing chamber, any remaining substance in the needle was discarded, the outside of the needle was rinsed, and the interior of the needle was flushed with fresh dimethyl sulfoxide before the needle was moved by the robotic arm to the next open vial (containing the next substance to be tested). With this device, small quantities of the various substances built up outside the end of the needle to form hard deposits, and it is believed that these deposits in some cases adversely affect the solubility determinations (e.g., the deposits may have “sponged up” subsequent test substances as they were being released from the needle, thereby preventing accurate determination of exactly how much of each substance had been added to the test mixture). The equilibration time (i.e., the time allowed for any entrained gas bubbles to leave the liquid in the cuvette before turbidity measurement) was 60 seconds. The assay has since been used internally by the present assignee but with a test tube instead of the cuvette as the testing chamber. Some of the foregoing is described in the 1997 Lipinski et al. article cited above, which indicates at page 17 that glass test tubes as small as 110 millimeters by 12 millimeters can be used by the Hach turbidimeter but that “[t]he use of even smaller tubes and the resultant advantage of reduced volume is precluded by light scattering from the more sharply curved surface of a smaller diameter tube.”

In short, there are still no satisfactory methods that are rapid, accurate, have good reproducibility, and require essentially no attention (i.e., human operator attention) for screening large numbers of substances to determine their kinetic solubility (particularly aqueous kinetic solubility), e.g., because they are being considered for as candidates for orally administered drugs. Moreover, there is no satisfactory apparatus for carrying out such methods. Accordingly, the need exists for such methods and apparatus and the need is increasing and becoming more urgent as combinatorial chemistry methods produce ever increasing numbers of compounds and the proportion of less soluble compounds in the population of compounds to be screened increases. Furthermore, the need exists for such methods and apparatus that also protect operators from contact with the substances to be screened and that require as little as possible of the substance for screening.

Straight septum-piercing needles are known (e.g., the needles used by medical professionals to withdraw fluids from vials for injection into patients). It is also known to put straight metal sleeves around such needles to protect them. In some cases, those metal sleeves have been close fitting. Such sleeves are available from Gilson, Inc. (Middleton, Wis., United States).

### BRIEF SUMMARY OF THE INVENTION

An invention that satisfies those needs and provides still other benefits that will be apparent to those skilled in the art has now been developed. Broadly speaking, in one aspect this invention concerns an automated kinetic solubility assay apparatus for assessing the kinetic solubility of one or more substances in one or more test fluids, the apparatus comprising:

(a) a cuvette for automatically receiving a first test fluid and a first substance, the cuvette having at least two spaced wall sections;

(b) test fluid addition means for automatically adding the first test fluid to the cuvette and substance addition means for automatically adding the first substance to the cuvette to cause initial contact of the first test fluid and first substance in the cuvette, thereby to produce a first testable mixture;

(c) turbidity measurement means for automatically measuring the turbidity of the first testable mixture in the cuvette using energy that is directed to pass through at least one of the two spaced wall sections of the cuvette, then the first testable mixture in the cuvette, and then through the other of the two spaced wall sections of the cuvette;

(d) kinetic solubility assessing means for automatically assessing the kinetic solubility of the first substance in the first test fluid from the turbidity of the first testable mixture in the cuvette; and

(e) removal means for automatically removing the first testable mixture from the cuvette.

In another aspect, the invention concerns an automated kinetic solubility assay apparatus for assessing the kinetic solubility of one or more substances in one or more test fluids, the apparatus comprising:

(a) a cuvette for automatically receiving a first test fluid and a first substance, the cuvette having at least two spaced wall sections;

(b) test fluid addition means for automatically adding the first test fluid to the cuvette and substance addition means for automatically adding the first substance to the cuvette, thereby to produce a first testable mixture;

(c) turbidity measurement means for automatically measuring the turbidity of the first testable mixture in the cuvette using energy that is directed to pass through at least one of the two spaced wall sections of the cuvette, then the first testable mixture in the cuvette, and then through the other of the two spaced wall sections of the cuvette;

(d) kinetic solubility assessing means for automatically assessing the kinetic solubility of the first substance in the first test fluid from the turbidity of the first testable mixture in the cuvette;

(e) removal means for automatically removing the first testable mixture from the cuvette; and

(f) cleaning means for automatically cleaning the cuvette to increase the transmittance of energy that can pass through at least its two spaced wall sections.

In another aspect, the invention concerns an automated kinetic solubility assay apparatus for assessing the kinetic solubility of one or more substances in one or more test fluids, the apparatus comprising:

(a) a cuvette for automatically receiving test fluids and substances, the cuvette having at least two spaced wall sections;

(b) test fluid addition means for automatically adding a first test fluid to the cuvette;

(c) substance addition means for automatically adding a first substance to the cuvette to cause in the cuvette initial contact of the first substance with the first test fluid, thereby to produce a first testable mixture, the substance addition means comprising means for automatically repeatedly adding the first substance to the cuvette;

(d) turbidity measurement means for automatically measuring the turbidity of the first testable mixture in the cuvette using energy that is directed to pass through at least one of the two spaced wall sections of the cuvette, then the first testable mixture in the cuvette, and then the other of the two spaced wall sections of the cuvette, the turbidity measurement means comprising means for automatically measuring the turbidity of the first testable mixture in the cuvette after each addition of the first substance to the cuvette;

(e) means for automatically halting the repeated addition of the first substance to the cuvette after the earlier of either of two conditions occurs: (i) the number of additions of the first substance to the cuvette exceeds a predetermined value or (ii) the turbidity of the first testable mixture in the cuvette exceeds a predetermined value;

(f) kinetic solubility assessing means for automatically assessing the kinetic solubility of the first substance in the first test fluid from the turbidity of the first testable mixture in the cuvette;

(g) removal means for automatically removing the first testable mixture from the cuvette;

(h) rinsing means to automatically rinse the cuvette after the first testable mixture has been removed from the cuvette by the removal means;

(i) transmittance determining means for determining the transmittance of energy that can pass through at least the two spaced wall sections of the cuvette after the first testable mixture has been removed from the cuvette by the removal means;

(j) cleaning means for automatically cleaning the cuvette to increase the transmittance of energy that can pass through at least its two spaced wall sections; and

(k) cleaning activation means to automatically activate the cleaning means to automatically clean the cuvette to increase the transmittance of energy that can pass through at least its two spaced wall sections if the transmittance determined by the transmittance determining means after the first testable mixture has been removed from the cuvette by the removal means is below a predetermined value.

In some preferred embodiments, the apparatus further comprises means to cause: (a) the test fluid addition means to automatically add a second test fluid to the cuvette and the substance addition means to automatically add a second substance to the cuvette to cause initial contact of the second test fluid and second substance in the cuvette, thereby to produce a second testable mixture, the addition of the second test fluid and second substance to the cuvette occurring after the cuvette has been rinsed;

(b) the turbidity measurement means to automatically measure the turbidity of the second testable mixture in the cuvette using energy that is directed to pass through at least one of the two spaced wall sections of the cuvette, then the second testable mixture in the cuvette, and then through the other of the two spaced wall sections of the cuvette;

(c) the kinetic solubility assessing means to automatically assess the kinetic solubility of the second substance in the second test fluid from the turbidity of the second testable mixture in the cuvette; and

(d) the removal means to automatically remove the second testable mixture from the cuvette.

In some preferred embodiments, the apparatus further comprises means for determining the transmittance of energy passing through at least the two spaced wall sections of the cuvette in the absence of the first (or second, or third, or subsequent) substances; cleaning means for automatically cleaning the cuvette to increase the transmittance of energy that can pass through at least its two spaced wall sections; and cleaning activation means to activate the cleaning means to automatically clean the cuvette to increase the transmittance of energy that can pass through at least its two spaced wall sections if the transmittance in the absence of any of the first, second, third, or subsequent substances is below a predetermined value. In some preferred embodiments (i) the substance addition means comprises means for automatically repeatedly adding the first (or second, or third, or subsequent) substance to the cuvette and/or the test fluid addition means comprises means for automatically repeatedly adding the first (or second, or third, or subsequent) test fluid to the cuvette and (ii) the turbidity measurement means comprises means for automatically measuring the turbidity of the first (or second, or third, or subsequent) testable mixture in the cuvette after each addition of the first (or second, or third, or subsequent) substance to the cuvette and/or after each addition of the first (or second, or third, or subsequent) test fluid to the cuvette. In some preferred embodiments, the apparatus further comprises means for automatically halting the repeated addition of the first (or second, or third, or subsequent) substance to the cuvette and/or the repeated addition of the first (or second, or third, or subsequent) test fluid to the cuvette after the earlier of either of two conditions occurs: (i) the number of additions of the first (or second, or third, or subsequent) substance or the first (or second, or third, or subsequent) test fluid to the cuvette exceeds a predetermined value or (ii) the turbidity of the first (or second, or third, or subsequent) testable mixture in the cuvette is above or below a predetermined value. In some preferred embodiments, a series of different test substances will be tested using the same test fluid (e.g., a fluid that adequately simulates normal digestive fluid).

As used herein, “cuvette” should be broadly understood and refers to a small laboratory vessel, which may have any size, shape, design, or material of construction that allows the benefits of this invention to be achieved. Preferred cuvettes are further described below.

“Substance” (also referred to as the “test substance”) means any substance whose kinetic solubility is to be assessed in a test fluid using embodiments of this invention. In a series of runs being made on a number of substances, the “first substance” and “second substance” will usually be different but in some cases may be the same (e.g., if replicate runs are being made).

“Test fluid” means any fluid in which the kinetic solubility of a substance is to be assessed using embodiments of this invention. In a series of runs being made on a number of substances, the “first test fluid” and the “second test fluid” will usually be the same but in some cases may be different.

“Test fluid addition means” refers to any structure (e.g., device) that can automatically introduce the test fluid into the testing chamber (e.g., the preferred cuvette).

“Substance addition means” refers to any structure (e.g., device) that can introduce the substance into the testing chamber (e.g., the preferred cuvette).

“Initial contact” of a substance and a test fluid means the first contact between the substance and the test fluid, which in the kinetic solubility assay of this invention occurs in the testing chamber, i.e., desirably the preferred cuvette.

“Testable mixture” (also referred to as the “test mixture”) means the mixture (regardless of the number of phases, i.e., whether a true solution (with one phase) or a mixture (having, e.g., a liquid phase and a solid phase)) formed by mixing the test fluid and the substance whose kinetic solubility is to be determined. The “first testable mixture” and the “second testable mixture” will usually have different compositions (e.g., because the substance in the two mixtures is different) but in some case may be the same.

“Spaced wall sections” refers to distinct wall sections that are spaced apart, whether they are on a single portion of a wall or are on different portions of a wall, e.g., on two spaced apart, oppositely disposed, parallel portions of the wall of a vessel (e.g., a cuvette) that has a square lateral cross-section.

“Turbidity measurement means” refers to any structure (e.g., device) capable of automatically measuring the turbidity of a fluid (e.g., a testable mixture or a test fluid in the absence of the test substance) by passing energy through the fluid and may, for example, be or include a nephelometer.

“Kinetic solubility assessing means” refers to any structure (e.g., device) for automatically evaluating the kinetic solubility of a substance and may include electronic circuitry and/or a programmable or programmed computer.

“Removal means” refers to any structure (e.g., device) for automatically removing fluids from the testing chamber (e.g., the cuvette).

“Rinsing means” refers to any structure (e.g., device) that can be used to rinse the testing chamber (e.g., cuvette) to less aggressively remove some or all of any residue of substances, test fluids, and/or test mixtures from the wall portions of the cuvette.

“Cleaning means” refers to any structure (e.g., device) that can be used to more aggressively remove some or all of any residue of substances, test fluids, and/or test mixtures from the wall portions of the cuvette. In some apparatus of this invention, much of the same structure may be used to clean and to rinse the cuvette and the difference between rinsing and cleaning may be the composition and/or number of fluids used for each operation (i.e., the rinsing or the cleaning) and/or the conditions under which those fluids are contacted with the inside of the cuvette (e.g., the number of times and/or force with which the fluids are moved into the cuvette, the force with which the fluids are moved around inside the cuvette, the temperature at which such contact occurs).

“Means for automatically halting repeated addition of the first substance to the cuvette” refers to any structure (e.g., device) that stops the repeated addition of the first substance to the cuvette and may include electronic circuitry and/or a programmable or programmed computer.

“Transmittance determining means” refers to any structure (e.g., device) that can determine the transmittance of energy that can pass through at least the two spaced wall sections of the cuvette and may include electronic circuitry and/or a programmable or programmed computer.

“Cleaning activation means” refers to any structure (e.g., device) that can automatically activate the cleaning means to automatically clean the cuvette and may include electronic circuitry and/or a programmable or programmed computer.

Each of the first, second, third, etc. substances will typically be held in its own container, for example, for storage and/or for transport from the laboratory where each was made (e.g., by combinatorial chemistry or other methods) to a place where it is to be stored or to a place where it is to be tested using the apparatus of this invention. Typically, each test substance will be at least partially (and desirably completely) dissolved in a liquid medium to facilitate its transport and manipulation, and dimethyl sulfoxide (“DMSO”), which is a powerful solvent, is preferred. Each such mixture may be referred to as a “sample mixture,” a “sample solution,” or a “sample.”

For some preferred embodiments, each container has a pierceable septum (i.e., a septum composed of material capable of being penetrated by the septum-piercing needle), the cuvette comprises a pierceable septum, and the apparatus further comprises needle manipulation means and a septum-piercing needle having a passageway, the needle and needle manipulation means being for (i) piercing the septum of the container with the needle, (ii) withdrawing the first substance from the container after the needle pierces the septum of the container and holding the withdrawn first substance in at least the passageway of the needle, (iii) withdrawing the needle from the septum of the container and piercing the septum of the cuvette with the needle, and (iv) discharging the withdrawn first substance from at least the passageway of the needle into the cuvette after the needle pierces the septum of the cuvette. To allow screening of two or more compounds at the same time, the apparatus preferably comprises a plurality of cuvettes, each cuvette being operatively associated with test fluid addition means, substance addition means, turbidity measurement means, and removal means.

A preferred cuvette of this invention (and preferably used with the apparatus of this invention) comprises:

(a) a bottom, a top, and a wall therebetween and connected to both, the bottom, the top, and the wall together defining an enclosed volumetric space for receiving fluid;

(b) a pierceable septum forming part of the top to allow fluid to be injected through the septum into the volumetric space within the cuvette;

(c) means to remove fluid from the volumetric space within the cuvette; and

(d) a vent fluidly communicating between the volumetric space and the region outside of the cuvette through which (i) gas (typically air) in the volumetric space in the cuvette can flow to the region outside the cuvette as fluid is injected into the volumetric space in the cuvette through the pierceable septum and (ii) gas (typically air) in the region outside of the cuvette can flow into the volumetric space in the cuvette when fluid is removed from the volumetric space in the cuvette.

In some embodiments, the wall of the cuvette comprises a curved surface, and/or a planar cross-section of the wall is circular, and/or the wall comprises at least three planar surfaces, and/or the wall comprises at least four surfaces, at least two of which are planar and are parallel to one another. In some embodiments, the means to remove fluid from the cuvette comprises one or more fluid ports at or near the bottom of the cuvette, and/or the vent is at or near the top of the cuvette, and/or the cuvette further comprises means at or near the bottom of the cuvette spaced from the septum to add fluid to the cuvette, and/or the volumetric space in the cuvette ranges in volume from 0.3 milliliters to 5 milliliters. In some embodiments, the cuvette further comprises means for agitating fluid in the cuvette and in some preferred embodiments, those means comprise a magnetic stir bar located inside the cuvette and at or near the bottom of the cuvette.

As noted above, each test substance is typically held in a container (e.g., a vial) and from there it must be transferred to the cuvette to be mixed with the test fluid (and in which further additions of test substance or test fluid are made). Each of the containers and cuvette is typically closed, for safety and other reasons, but each desirably is provided with its own pierceable septum. “Septum” refers to a thin partition that separates the contents of a container (in this case, the test substance in a vial or the testable mixture inside the cuvette) from the environment. For example, there is usually a septum at the top of a vial holding a drug solution through which a medical professional can insert the end of the needle of a hypodermic syringe to allow withdrawal of some of the solution for administration to a patient.

In the present invention, means are preferably provided to withdraw the test substance from its container through the septum of the container and introduce it into the cuvette through the septum of the cuvette. Thus, in another aspect, the apparatus further comprises needle manipulation means and a septum-piercing needle having a passageway, the needle and needle manipulation means being for (i) piercing the septum of the container with the needle, (ii) withdrawing the first substance from the container after the needle pierces the septum of the container and holding the withdrawn first substance in at least the passageway of the needle, (iii) withdrawing the needle from the septum of the container and piercing the septum of the cuvette with the needle, and (iv) discharging the withdrawn first substance from at least the passageway of the needle into the cuvette after the needle pierces the septum of the cuvette.

A preferred septum-piercing needle of this invention (which needle is preferably used with the apparatus of this invention) comprises a straight upper portion, a curved lower portion, a longitudinal axis, a piercing end at the end of the curved lower portion, and a non-piercing end in the straight upper portion; the needle comprising a rigid exoskeleton lined with a corrosion-resistant cannula having a central elongate passageway running from the piercing end of the needle to the non-piercing end of the needle, the passageway of the cannula being adapted to hold fluid and terminating at the piercing end of the needle in an opening, the piercing end of the needle being adapted for piercing the pierceable septum of a container holding fluid (e.g., fluid comprising a test substance) to allow fluid to be withdrawn from the container and to flow through the opening of the passageway at the piercing end of the cannula into the passageway of the cannula, the plane of the opening of the passageway at the piercing end of the needle being substantially parallel to the longitudinal axis of the straight upper portion of the needle.

In some embodiments of the septum-piercing needle of this invention, the passageway of the cannula has an average diameter of from 100 to 300 microns and a length of from 10 to 150 millimeters, and/or the exoskeleton is made of metal, and/or the cannula is made of glass.

As discussed above, one problem with suggested turbidimetric kinetic solubility devices arises from adhesion of substances (e.g., particularly of gummy or tar-like substances) to the walls of vessels used during turbidimetric analysis. As also noted, this seems to be exacerbated by the apparent trend for an increasing proportion of the new substances that are to be screened to have lower and lower aqueous solubility and to be more and more gummy or tar-like. Thus, in one aspect of this invention, the previously used separate mixing chamber (for mixing the test fluid and the substance being screened) has been eliminated and the first (initial) contact of the test substance and the test fluid occurs in the vessel in which the turbidimetric measurements will be made (i.e., the cuvette). As noted above, one feature of this invention provides for monitoring the cleanliness of the cuvette by determining if the transmittance of energy through the wall of the cuvette in the absence of any test substance falls below a predetermined minimum (which is the same as determining whether the absorbance rises above a predetermined maximum). If it does, the cuvette is cleaned, in accordance with another aspect of this invention and preferably using a cleaning agent that has been found to be particularly effective. That cleaning agent comprises a mixture of ethylenediaminetetraacetic acid (“EDTA”) and glass cleaner, typically 0.001% w to 50% w EDTA and desirably 0.001% w to 25% w EDTA. In some preferred embodiments, the glass cleaner comprises water, ammonium hydroxide, 2-propanol, 2-butoxy ethanol, and anionic surfactant (preferably sodium dodecylbenzenesulfonate). The cleaning agent may be used as part of a cleaning mixture that also contains dimethyl sulfoxide. Thus, another aspect of the invention concerns a cleaning mixture comprising at least 1% w (preferably at least 10% w) dimethyl sulfoxide and at least 1% w (preferably at least 10% w) of the cleaning agent.

As noted above, thermodynamic solubility is typically measured after solute and solvent have been in contact for at least 24 hours. As far as is known to applicant, there is no industry-wide period of contact used (i.e., there is no standard period) for measuring kinetic solubility. As used herein, the term “kinetic solubility” refers to the amount of a substance that will dissolve in a test fluid under given conditions (e.g., temperature, pressure, and agitation) in a period of time less than that required for equilibrium to be reached. That period will usually be less than 24 hours, desirably less than an hour, more desirably less than 30 minutes, most desirably less than 15 minutes, preferably less than 10 minutes, more preferably less than 5 minutes, and most preferably less than 2 minutes. In other words, with the apparatus of this invention, the period of time can be exceedingly short, thereby allowing a large number of compounds to be screened in a day, especially if the apparatus has a plurality of testing chambers (e.g., the preferred cuvette). With the preferred procedure and kinetic solubility assay apparatus described herein, which has only one testing chamber (i.e., cuvette), approximately 400 compounds (substances) can be screened for aqueous solubility in a 72-hour period, which is an average of just under 11 minutes per compound, far faster than with the earlier assay.

The high-throughput solubility screening achievable with the apparatus of this invention is made possible in part by the fact that the apparatus is automated. As used herein, the terms “automated,” “automatically,” and the like refer to the fact that the apparatus, under normal operating conditions and once it is stocked or supplied with reservoirs or sources of the one or more test fluids and other fluids used, a source of the one or more test substances (e.g., in containers such as small vials), etc., and is properly programmed and set (e.g., told how many sample vials it is to process), is capable of carrying out the intended process to completion (i.e., the determination of the kinetic solubility of all of the test substances) without the need for human intervention after the process has been initiated.

Another feature of this invention is the accuracy and reproducibility of measurement. That is made possible in part by the accuracy with which fluids are metered into the testing chamber (e.g., cuvette), which in turn is made possible in part by the septum-piercing needle of this invention, which does not allow build-up of any hard deposits, and the combination of needle, pump, controller, etc., which together so accurately meter the test substance into the cuvette (in a preferred embodiment, the aliquots of test substance added to the cuvette by the needle have a volume of only 0.5 microliters and the coefficient of variation in volume size is less than 5% from aliquot to aliquot).

Other features of this invention that help provide the accuracy and reproducibility are the means that ensure that substances previously present in the testing chamber (e.g., the cuvette) have not dirtied the wall of the chamber enough to significantly lower the transmittance of energy (e.g., light) passing through the wall and thereby adversely affect the transmittance data. Those means include means for determining the transmittance of energy passing through the wall (e.g., the two spaced wall sections) of the cuvette in the absence of the first substance and/or after the testable mixture has been removed from the cuvette (e.g., when only test fluid, such as simulated gastrointestinal fluid, is present), means for automatically cleaning the cuvette to increase the transmittance of energy that can pass through the wall (e.g., the two spaced wall sections), and cleaning activation means, which activate the cleaning means to automatically clean the cuvette to increase the transmittance of energy that can pass through the wall (e.g., the two spaced wall sections) if the transmittance in the absence of the first substance and/or after the testable mixture has been removed from the cuvette is below a predetermined value. It will be understood by one skilled in the art that, in the context of this invention, a lower transmittance is equivalent to a higher turbidity. The accuracy and reproducibility of measurement is also made possible in part by the cleaning solution of this invention, which solution has been found to be particularly effective in removing (in an automated step) material (e.g., a gummy residue from a test substance that had previously been in the test chamber) from the surface (e.g., wall) of the test chamber (e.g., cuvette) that otherwise is difficult to remove without operator intervention (e.g., scrubbing with a brush). Other features of the invention that help provide the accuracy and reproducibility of measurement will be apparent to those skilled in the art.

The kinetic solubilities likely to be encountered using the preferred apparatus and assay of this invention (in micrograms of substance per milliliter of testable mixture, the majority of which testable mixture is the test liquid, e.g., a liquid that simulates gastric juices (pH of 1.2) or an aqueous pH 7 chloride-free buffered solution), and the characterizations of those kinetic solubilities, are as follows:

| Kinetic Solubility (μg/ml) | Characterization |
|---|---|
| <5 | Poor |
| 5 to 25 | Borderline |
| 25 to 65 | Acceptable |
| >65 | Very Good |

It has been found that turbidimetric kinetic solubilities less than about 30 micrograms per milliliter usually exceed thermodynamic values by 2 to 4 times and that turbidimetric kinetic solubilities are comparable to thermodynamic solubilities for kinetic solubilities greater than about 50 micrograms per milliliter (with certain exceptions discussed below); however, the kinetic solubilities determined using this invention are sufficiently, consistently, and reproducibly representative of the true equilibrium solubilities to make use of these kinetic solubilities highly advantageous, particularly in the early stages of drug candidate screening. Thus, the kinetic solubility of a test substance in a test fluid determined using the apparatus and assay of this invention is obtained in a matter of minutes (rather than the dozens of hours required for true equilibrium solubility determination) and gives a sufficiently accurate indication of the relative true equilibrium solubility to allow discriminating among the numerous compounds being screened, e.g., so that compounds having poor kinetic solubility can be eliminated from further consideration (because they will have undesirably low equilibrium solubility) and compounds having very good kinetic solubility can be considered more closely (because they will have desirably high true equilibrium solubility).

In summary, the present invention provides apparatus that determines kinetic solubility rapidly, accurately, with good sensitivity, and with good reproducibility, that is automatic, requiring essentially no operator attention, that provides for increased safety (e.g., by reducing the risk of the operator's contacting the test substances and any carrier fluids, through use of septum-sealed containers and test chambers and use of the septum-piercing needle), that is reliable, that requires only very small amounts of test substances, that overcomes the problems associated with a small diameter round cuvette and with a square lateral cross-section cuvette (this is further discussed below), and that can screen large numbers of substances to determine their kinetic solubility with all of the above-noted advantages. The present invention also provides a cuvette, a needle, and cleaning solutions, each having its own features and advantages and each of which may be used in or with the automated kinetic solubility assay apparatus of this invention. Other features and advantages of the various aspects of the invention should be apparent to those skilled in the art.

### BRIEF DESCRIPTION OF THE DRAWINGS

To facilitate further discussion of the invention, the following drawings are provided in which:

FIG. 1 illustrates a preferred automated kinetic solubility assay apparatus of this invention, part of which apparatus is a turbidimeter;

FIG. 2 shows the principle components of two fluid handling systems connected to the preferred cuvette and the principle components of one fluid handling system connected to the preferred septum-piercing needle;

FIG. 3 shows the principle components of one fluid handling system connected to the preferred needle rinse chamber for the septum-piercing needle;

FIG. 4 comprises three parts, FIGS. 4A, 4B, and 4C, which together are a block diagram showing the principle steps of a preferred kinetic solubility assay, which may be practiced with the apparatus of FIG. 1;

FIG. 5 illustrates a preferred cuvette with a micro stir-bar inside at the bottom and without any liquid inside;

FIG. 6 is the same as FIG. 5 but with the cuvette partially filled with liquid;

FIG. 7 is the same as FIG. 5 but with the cuvette filled with the maximum amount of liquid it would normally contain;

FIG. 8 is a front elevational view of a preferred holder used in the apparatus of FIG. 1 for the preferred cuvette showing the inlet for the light that is preferably beamed into the cuvette for determining the turbidity of the fluid in the cuvette;

FIG. 9 is a rear elevational view of the cuvette holder of FIG. 8 showing the rear outlet through which light introduced through the inlet can exit the holder for detection for determining the turbidity of the fluid in the cuvette;

FIG. 10 is a left side elevational view of the cuvette holder of FIG. 8 showing the left outlet through which light introduced through the inlet can exit the holder for detection for determining the turbidity of the fluid in the cuvette;

FIG. 11 is a top (or plan) view of the cuvette holder of FIG. 8 containing the cuvette of FIG. 5;

FIG. 12 shows the cross-section of a cuvette of this invention as viewed from the top comprising a circular wall;

FIG. 13 shows the cross-section of a cuvette of this invention as viewed from the top having a wall comprising three planar surfaces;

FIG. 14 is shows the cross-section of a cuvette of this invention as viewed from the top having a wall comprising a non-circular curved wall;

FIG. 15 shows the cross-section of a cuvette of this invention as viewed from the top having a wall comprising two planar surfaces that are planar and parallel to one another;

FIG. 16 is a perspective view of a preferred septum-piercing needle of this invention;

FIG. 17 is an enlarged cross-sectional view of part of the lower curved portion of the septum-piercing needle of FIG. 16;

FIG. 18 is an enlarged view of the lower open end portion of the septum-piercing needle of FIG. 16;

FIG. 19 is a block diagram showing the main cards, interfaces, and computer that provide for automatic control of the turbidimeter of the apparatus of FIG. 1;

FIG. 20 shows the layout of the custom turbidimeter interface board, including ten similar integrated circuits (U1 through U10), each of which decodes information concerning one of ten important states of the turbidimeter so that the information can be sent to the computer;

FIG. 21 comprises two parts, FIGS. 21A and 21B, which are schematics of circuitry on the custom turbidimeter interface board;

FIG. 22 is a two-part table (i.e., FIGS. 22A and 22B) setting forth the bus definitions of the custom turbidimeter interface card (board), the first part of which table (FIG. 22A) shows the correspondence between the signals from the turbidimeter that normally appear on LED (light-emitting diode) displays, the hardware and characteristics of the custom turbidimeter interface board, and the 32-channel digital I/O board (or card), and the second part of which table (FIG. 22B) shows the correspondence between some of the hardware of the custom turbidimeter interface board, the 32-channel relay board (or card), and some of the hardware and functions of the turbidimeter;

FIG. 23 is the schematic of a circuit used with integrated circuit (“IC”) U6, a typical operational amplifier (op-amp) circuit comprising a peak detector, a comparator, a low-pass filter, and unity gain followers (buffers) and located on the custom turbidimeter interface board (the same circuit arrangement is used with all ten integrated circuits, U1 through U10);

FIG. 24 is a table setting forth the correspondence between the switch controls on the turbidimeter and the 32-channel relay card (board);

FIG. 25 is graph of NTU units plotted against addition number, which shows the mean average increase in NTU reading for a mixture of test fluid and 1000 NTU standard for each 0.5 microliter addition of 1000 NTU standard to the test fluid, where twelve replicate runs were made and eight additions were made to each of those identical test fluid aliquots;

FIG. 26 is graph of baseline NTU (Nephelometric Turbidity Units) values plotted against sample number, which illustrates possible baseline readings (i.e., NTU values for the test fluid in the cuvette prior to test substance addition) for the different samples as compared to an NTU value designated to be “high” (approximately 0.092, as shown by the shorter broken lines) and an NTU value designated as a “maximum” allowable (approximately 0.11, as shown by the longer broken lines); and

FIG. 27 is a graph of maximum, mean, and minimum kinetic solubility readings for fourteen different test substances.

These drawings are for illustrative purposes only and should not be used to unduly limit the scope of the present invention.

### DETAILED DESCRIPTION OF THE INVENTION

The present invention provides apparatus that can perform an automated kinetic solubility assay of one or more substances in one or more test fluids. Additionally, the present invention provides for a cuvette, a septum-piercing needle, and cleaning solutions, each of which may be used alone or in combination as part of or with the claimed apparatus. The automated kinetic solubility assay apparatus of the present invention has various fluid storage and handling devices (e.g., reservoirs, syringes, valves, and tubing) that are operatively connected to the cuvette and are monitored and/or controlled by a computer. Data obtained by the apparatus regarding assessed kinetic solubility, cuvette cleanliness, etc. may be automatically stored in memory or exported for use in other devices or computer programs (e.g., a spreadsheet or a database, such as an Oracle database).

The invention can be used to assess the kinetic solubility of various substances (e.g., drug candidate compounds) in one or more test fluids (e.g., pH-buffered solutions). Preferred substances are drugs, drug candidate compounds, excipients, excipient candidates, and combinations thereof; however, the various embodiments of the present invention may be used for any substance and their use is not limited to the field of drug discovery. Particularly preferred substances to be tested with the apparatus of this invention are drug candidate compounds for potential use as drugs in animals and particularly in humans. Substances to be tested may comprise a single compound or a combination of more than one compound. The substance to be tested may be in any form, e.g., a solid (e.g., a crystalline powder), liquid, emulsion, gum, wax, tar, staticy solid, glass, and any other form that cannot be weighed easily. The substance may be dissolved in a carrier fluid that is different from the test fluid. Dimethyl sulfoxide (“DMSO”), which is a powerful solvent, is a preferred carrier fluid. Methanol and acetonitrile may also be used as the carrier fluid. Carrier fluids are preferred because they facilitate transport of the test substances and they facilitate testing of the substances when using automatic testing apparatus.

Test fluids may be one or more aqueous liquids, one or more organic liquids, or mixtures thereof. The test fluids may be single phase or multi-phase and may be in any form (e.g., an emulsion). For screening drug candidates, preferred test fluids include body fluids and fluids that mimic the properties of body fluids. Particularly preferred test fluids are aqueous pH-buffered solutions, stomach acid, bile, blood, blood plasma, sinovial fluid, saliva, mucous, and any other biological fluid. If a compound being screened releases chloride ions in the test fluid and the test fluid by itself also contains chloride ions, the solubility reported would likely be too low because of a common ion effect; however, the use of non-chloride containing pH 7 phosphate aqueous buffer (which is preferred when screening drug candidates for oral administration) approximates physiological pH and avoids reporting low solubilities for hydrochloric acid salts or amines. For amine HCI salts, solubility is depressed by the chloride common ion effect; however, in a drug discovery program, this can be solved by changes in the salt form in other (later) stages of the development process.

The kinetic solubility assay apparatus of this invention assesses the kinetic solubility of a substance by detecting the amount of, and determining changes in the amount of, suspended particles in the test mixture, which is also referred to herein as a “testable mixture.” The “test mixture” or “testable mixture” comprises the substance (or substances) to be tested or assessed (i.e., the solute) and the test fluid (i.e., the solvent). The testable mixture will also contain the carrier fluid (e.g., DMSO) in which the test substance or substances has or have been transported, if a carrier fluid is used. Kinetic solubility may be indicated by the point at which a substance precipitates out of a test mixture: repeated addition of the substance to the test chamber (e.g., cuvette) saturates the test fluid in the test chamber and at some point, addition of more of the substance to the test mixture results in formation of a solid phase, yielding a suspension (the “concentration method”). Kinetic solubility may also be indicated by the point at which a suspension introduced into the test fluid in the test chamber becomes a solution: repeated addition of the test fluid (solvent) allows the solid phase (i.e., comprising the substance being tested) to dissolve into the additional test fluid (the “dilution method”).

When using the dilution method, the test mixture starts as a saturated solution/solid phase mixture in which particles of the compound are suspended. A known quantity of test fluid is added to dilute the test mixture. The test mixture is then examined for the presence of suspended particles. If they are present, another known quantity of test fluid is added and the test mixture is examined for the presence of suspended particles. This procedure is repeated until suspended particles are no longer detected (or the maximum number of additions of test fluid has been made), at which point a kinetic solubility is assessed. Turbidimetric means may be employed to detect the presence or absence of particles in the test mixture.

Using the concentration method, a known quantity of a substance is added to a known quantity of test fluid with mixing to form a test mixture. After a short period of time, the test mixture is examined for the presence of suspended particles. If none are present, another known quantity of the substance is added and the test mixture is again examined for the presence of suspended particles. This procedure is repeated until suspended particles are detected (or the maximum number of additions of substance has been made), at which point a kinetic solubility is assessed. Again, turbidimetric means may be used to detect the absence or presence of particles in the test mixture.

Either method may be practiced with the claimed apparatus but the concentration method is preferred, for a number of reasons. With the concentration method, one need not start with a suspension (i.e., a saturated solution of the test substance containing in addition a solid phase of the substance to be tested), as is required with the dilution method. Preparation of the initial suspensions may itself be time-consuming and complex (because, e.g., different substances to be tested may have widely varying solubilities in the test fluid). Furthermore, with the concentration method, one can start with a standard amount of test substance in carrier fluid for all of the substances to be tested (e.g., 10, 20, or 40 micrograms of substance per microliter of carrier fluid), which simplifies and shortens the time required for preparation of substance/test fluid solutions.

Any means capable of detecting differences in the amount of energy (e.g., sound, light, heat) scattered or dissipated or absorbed by suspended particles in a liquid when energy passes through the liquid may be employed. Preferably, turbidimetric means are used and preferred turbidimetric means use filtered incandescent light. Preferably, the test fluid or test mixture is irradiated with a light beam incident on the test chamber (e.g., a cuvette) at a given location and readings are obtained from one or more optical sensors located at predetermined positions with respect to the point of entry of the incident beam (e.g., ninety degrees and one hundred eighty degrees from the point of entry of the light beam). The optical sensors measure the amount of light scattered and/or not scattered by the particles (if any) in the test fluid or test mixture. Optical sensors are well known in the art. Preferred sensors are available from UDT Sensors Inc. (Hawthorne, Calif., United States), Hamamatsu Photonics (Bridgewater, N.J., United States), and Centronic (Newbury Park, Calif., United States).

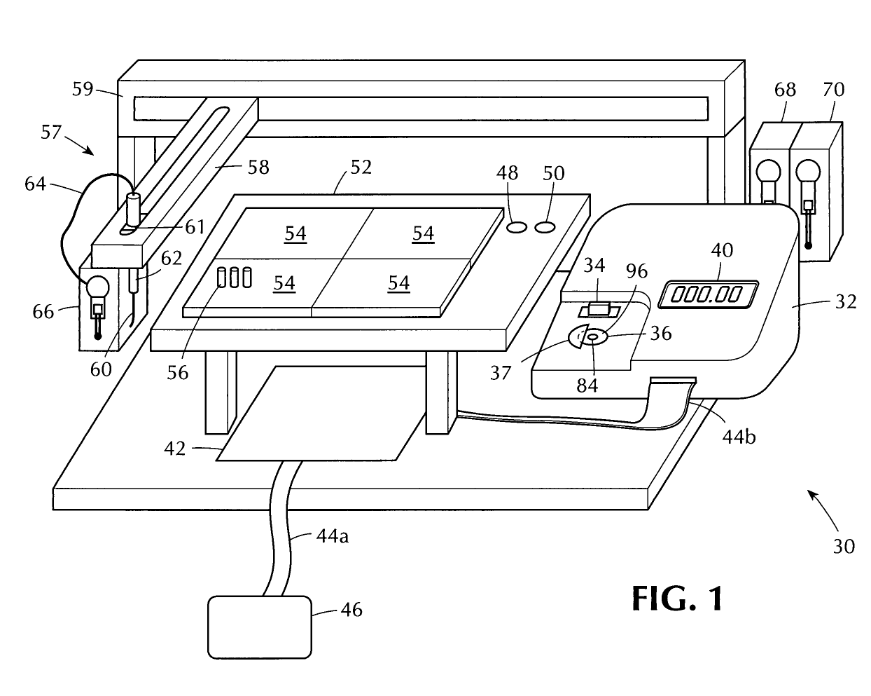

FIG. 1 shows preferred kinetic solubility assay apparatus 30 comprising preferred turbidity measurement means 32, namely, a modified Hach 2100N turbidimeter, which is marketed by Hach Company (corporate offices in Loveland, Colo., United States), which is a wholly-owned subsidiary of Danaher Corporation. Other turbidimeters can be used, e.g., HP Diode Array HP8452A (Hewlitt-Packard Corporation), HF Scientific Micro200 Turbidimeter (HF Scientific Inc., Fort Meyers, Fla., United States), as well as other similar turbidimeters known to those skilled in the art. The preferred Hach turbidimeter could not be used as is in the automated kinetic solubility assay of this invention and applicant had to make a number of modifications so that it could be employed.

As far as is known to applicant, commercially available turbidimeters are designed for measuring the turbidity of commercial manufacturing and waste, sewage, and similar streams, the turbidities of which are typically orders of magnitude higher than the turbidities to be encountered in the kinetic solubility assay of this invention. By way of background, cow's milk containing 2% fat has a turbidity reading of approximately 4000 NTU, skim cow's milk has a turbidity reading of approximately 2000 NTU, and the threshold turbidity value discernable by the naked human eye is approximately 20 NTU. Thus, the commercially available units are designed for working at high turbidities (e.g., hundreds or more NTU) but the turbidity values encountered in the kinetic solubility assay of this invention are far lower, generally not exceeding 10 NTU or even less (depending on the particular kinetic solubility assay). Furthermore, those commercially available devices are not designed to handle low volume samples.

As far as is known to applicant, commercial turbidimeters are not designed with any serious consideration given as to the amount of sample available for testing (because the size of the body of liquid whose turbidity is to be determined generally far exceeds the size of the sample needed by the turbidimeter), whereas the amount of a drug candidate available for screening is typically only a few milligrams at most. In other words, there is simply too little of the typical test mixture when the apparatus of this invention is used for kinetic solubility assays (particularly in drug discovery screening) to properly fill the test tubes or large cuvettes of the commercially available turbidimeters. As a result, the cuvette (testing chamber) containing the testable mixture (i.e., test fluid, substance to be tested, and any carrier fluid used) used in the apparatus of this invention must be significantly smaller than the test tube or large cuvette typically used in the commercially available devices (which test tube or large cuvette typically has a circular lateral cross-section). However, making a cuvette having a circular lateral cross-section smaller than the typical commercially available test tube or large cuvette (so that the smaller quantities of test mixture properly fill the cuvette) increases the scattering of light because of the significantly greater curvature of the wall of the smaller cuvette, and that in turn causes significantly and erroneously higher than actual turbidities to be reported with the smaller circular cuvette (the devices cannot determine the difference between scattering caused by particles in the test mixture and scattering caused by curvature of the cuvette wall).

The commercially available turbidimeters are also not automated and may be referred to as single-shot or single-reading units, that is, they are designed to allow an operator to place a test tube or large cuvette already containing the sample into the sample port of the device, take a turbidimetric reading of the sample, and remove the test tube or cuvette; they are not designed to allow a small partial or intermediate sample to be placed in the device and have a fluid repeatedly added to the contents of the cuvette (in this case, either the substance to be tested, if the concentration method is being used, or the test fluid, if the dilution method is being used). In other words, the commercially available turbidimeters contemplate insertion and removal of the testing chamber (the test tube or large cuvette) containing everything that is going to be tested for each reading and do not contemplate holding the cuvette in position in the device with only a partial or intermediate sample to facilitate repeated addition of the substance or the test fluid, as is preferably done with the preferred kinetic solubility assay apparatus of this invention. Furthermore, commercially available turbidimeters are not designed to keep the testing chamber in position after the sample is complete (i.e., all additions have been made), after all the intermediate and final readings have been made, and then to empty and clean the testing chamber while it is still in the device, as is preferably done with the preferred kinetic solubility assay apparatus of this invention.

In short, the design of the commercial devices (for high turbidity levels, large changes in turbidity, large amounts of testable quantities, and non-automatic functioning) make those devices unsuitable for the technological environment in which applicant is working and make those devices unusable in or with the present invention without the modifications made by applicant.

The modifications made to the Hach 2100N turbidimeter by applicant are directed toward overcoming these problems and include designing a small square lateral cross-section cuvette (to avoid the undesirably high scattering that would be caused by a small diameter circular lateral cross-section cuvette, such scattering preventing the obtention of reproducible results), designing a special cuvette holder to select only some of the possible outlet beams (i.e., beams exiting the cuvette) when using the smaller square lateral cross-section cuvette (to avoid the scattering caused by the corners of the square cross-section), modifying the turbidimeter to hold the cuvette in position (even when the septum-piercing needle that has been pushed in through the cuvette's septum to add test substance or test fluid into the cuvette is being pulled out through the septum), and adding hardware and circuitry (e.g., a custom turbidimeter interface board) to monitor and control the turbidimeter. Furthermore, applicant has calibrated the turbidimeter for the much smaller turbidities and changes in turbidity to be encountered in the technological environment of kinetic solubility assays. These modifications are described in detail below.

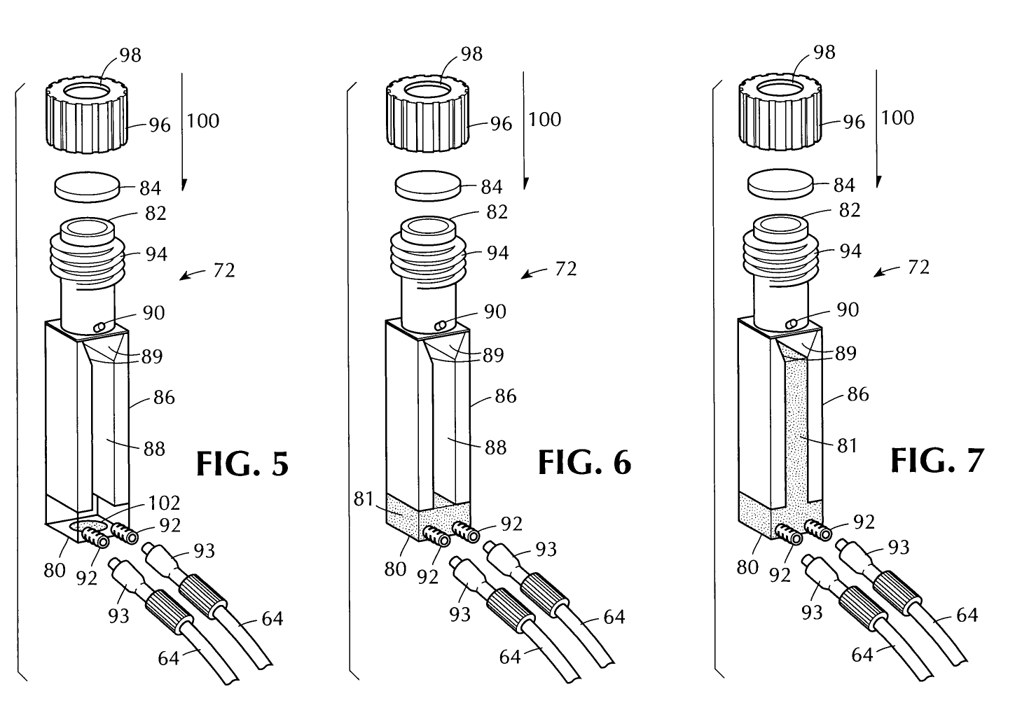

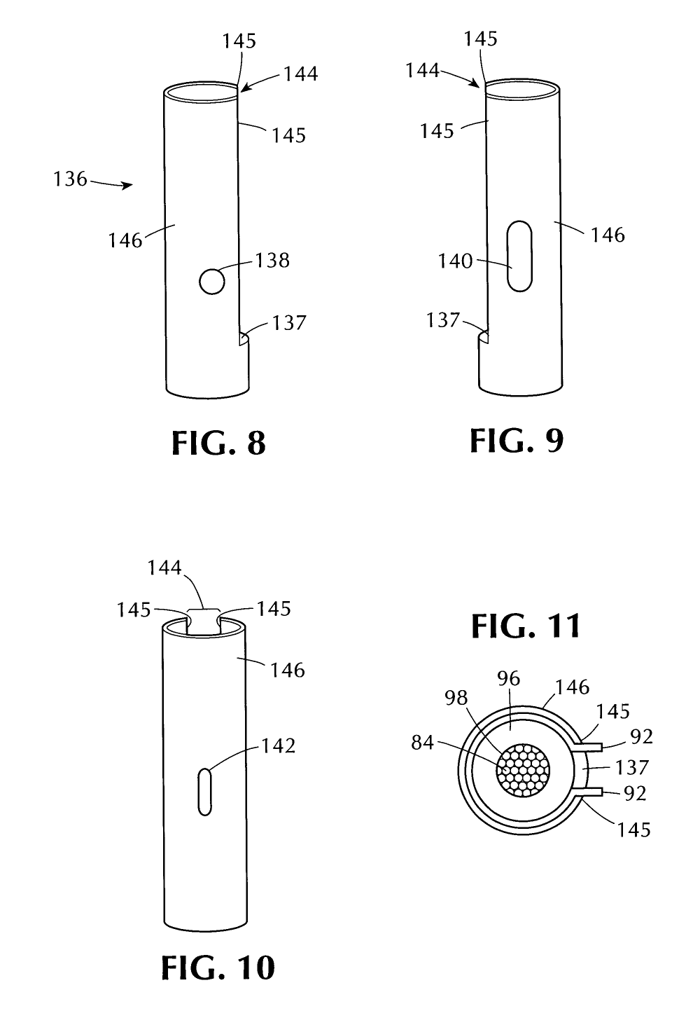

Modified turbidimeter 32 has display 40, on which various data (e.g., turbidimetric data) are displayed, and sample port 36, which provides an inlet for placing cuvette 72 (not shown; see, e.g., FIGS. 5 to 7) into special cuvette holder 136 (see, e.g., FIGS. 8 to 11), which is located within the body of the turbidimeter beneath the sample port. Cuvette 72 has cap 96 and pierceable septum 84 visible through central opening 98 in cap 96 (see, e.g., FIGS. 5 to 7).

Retainer 37 is attached to the outside of the device and partially blocks the opening of sample port 36 so that when needle 60 is pulled up through septum 84, cuvette 72 (possibly along with cuvette holder 136) is not pulled up out of the turbidimeter (which might occur because of the tight fit of the pierceable septum around the outer circumference of the needle). In this embodiment, retainer 37 cannot block the entire opening of port 36 because needle 60 must have access to septum 84 of cuvette 72. The needle is another aspect of the invention and is further described below.

Sample port 36 need not be of any particular shape or size as long as it allows the cuvette to be placed into the device (and preferably into the cuvette holder) and to be held firmly in place and also allows the septum-piercing needle to access the top of the cuvette (where the septum of the cuvette is desirably located). The sample port need not be equipped with a retainer, which may be in the form of a removable round cover, if other means are provided for keeping the cuvette (and cuvette holder) firmly in place, even when the needle is being withdrawn from the septum of the cuvette. The size, shape, and location of the sample port will vary according to the particular turbidimeter, cuvette holder, cuvette, etc. used. The design and material of construction of retainer 37 are not critical, and the retainer can be of any design and material of construction so long as it can perform its intended functions.

Turbidimeter 32 is equipped an incandescent light source (not shown) and with 650 nm (nanometer) light filter 34 (only the top handle of which is visible in FIG. 1), which filters the light before it is further used in the device (i.e., before it is incident upon wall 86 of the cuvette; see FIGS. 5 to 7). The preferred 650 nm filter is High Pass Filter P/N LPF-650 marketed by CVI Laser Corporation (Albuquerque, N. Mex., United States). Filter 34 allows only wavelengths of 650 nm and higher to pass. The filter blocks the passage of light in the visible spectrum, which runs from about 410 nm (violet) to about 650 (red), as well as light of wavelengths below the visible spectrum. Removal of wavelengths below 650 nm helps increase the accuracy of kinetic solubility determinations by reducing erroneous turbidity readings. Light having wavelengths below 650 nm can cause fluorescent emission (by compounds that become excited by wavelengths below 650 nm) and/or scattering (by compounds of certain colors), resulting in either case in erroneous turbidity readings. Depending on the energy type used to accomplish the turbidimetric analysis, various filters may be applied to one or more of the energy beams. Any filter or combination of filters known to those skilled in the art that is able to absorb the desired energy spectrum may be used, and one skilled in the art will know which filters to use with various energy sources (e.g., infrared light, laser light, polarized light).

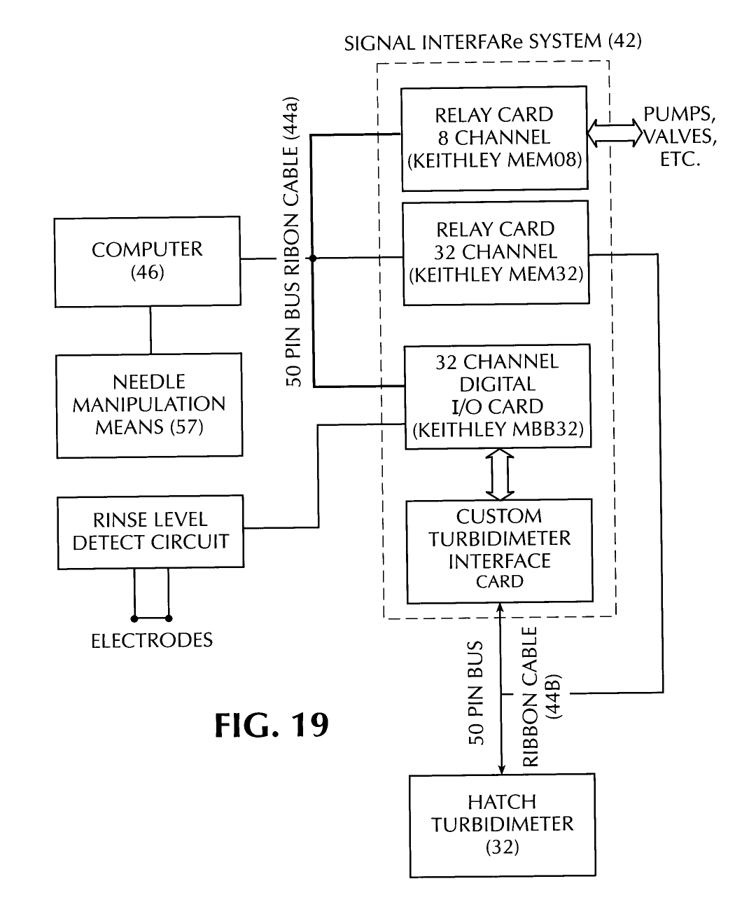

One set of modifications to the turbidimeter involves installing a 50-pin connector to the turbidimeter housing and adding various jumper wires to connect from the turbidimeter's standard circuitry to various pins on that connector. Fifty-pin connector cable 44b runs from the 50-pin connector installed by applicant in turbidimeter 32 to signal interface system 42, which in turn is connected by 50-pin connector cable 44a to computer 46 or to a computer network. Signal interface system 42 comprises two standard relay boards, one standard input/output board, and a custom turbidimeter interface board, all four of which boards are described below in connection with FIGS. 19 to 24 (in this context, “board” and “card” are used interchangeably). The design of signal interface system 42 is not critical and will vary with the particular apparatus used (e.g., turbidity measuring means (turbidimeter), needle manipulation means, valves, pumps, pump controllers). The signal interface system allows automated monitoring and control of all apparatus, preferably by a programmable computer.

Computer 46 may be any type of special purpose or general purpose computer (e.g., a desktop PC), which, among other things, receives data from the turbidimeter and other equipment, analyzes the data, controls the various pumps, valves, needle manipulation means, and the turbidimeter, and stores turbidity and other data. As previously indicated, a source code listing for the computer program is part of this application and appears below.

The substances to be tested are transported to the apparatus of this invention (e.g., from storage, from a synthesis laboratory) using any appropriate means, and sample vials 56 have been found to be particularly useful as transport containers. The design of those vials is not critical and may be of any suitable design. Sample vials are generally cylindrical in shape and have a volume of approximately 10 to 300 milliliters, are made of glass or plastic or a combination thereof, and have a pierceable septum at their top. The septum has approximately the shape of a squat cylinder, measuring about 1.5 millimeters thick and 8 millimeters in diameter, and is preferably made of rubber reinforced with plastic. The septum prevents the loss of substance and/or solvent from the vial and increases the safety of handling of chemical compounds (e.g., reduces the chance of an operator or other individual contacting the substances to be tested and/or the carrier fluid). Each of sample vials 56 is capable of holding a substance (or mixture of substances), which may be in liquid or solid form (e.g., powder, crystals) and which in any case may be in a carrier fluid (e.g., dissolved in DMSO). A preferred sample vial is a 0.1 milliliter (nominal volume) screw cap vial, which measures approximately 12 millimeters outer diameter by 32 millimeters in length, which may contain approximately 0.8 to 1.3 milligrams of test substance and up to 150 microliters of DMSO in a soft glass insert rigidly mounted within the clear plastic outer housing of the vial, and which is available from P.J. Cobert Associates, Inc. (St. Louis, Mo., United States). The septum of this preferred vial measures 1.5 millimeters high and 8 millimeters in diameter and is made of rubber and plastic.

Sample vial racks 54, which rest on support surface 52, are each capable of holding ninety-six sample vials 56. Means described below are used for transporting the substances to be tested from the vials to the cuvette. Use of four 96-vial sample racks 54 is preferred, which allows the apparatus to be “loaded” with three hundred eighty-four samples for kinetic solubility assay. Loading the apparatus may be as simple as placing sample racks 54 on surface 52 or may be more complex and/or automated, and the apparatus may be modified to process a greater or lesser number of sample vials. For example, a much larger number of vials could be positioned in a robotic system for feeding one vial to surface 52 or rack 54 or some other rest area every few minutes. A conveyor system could bring the sample vials one at a time from storage, where thousands of sample vials could be held. For the sake of clarity, only three sample vials 56 are shown in FIG. 1 but the apparatus will typically have far more sample vials loaded on the four sample vial racks 54.

Once sample vials 56 are in position, the substance inside each vial is transferred (along with any carrier liquid) to the testing chamber. The substances may be tested one at a time (e.g., if there is only one testing chamber, i.e., cuvette) or a plurality may be tested at the same time (e.g., if there are separate cuvettes for each substance). In the latter case, a single turbidimeter may be used or several may be arranged in parallel so that two or more of the plurality of substances may be tested at the same time. A single turbidimeter may have a carousel or other arrangement to bring one cuvette at a time from a plurality of cuvettes into position for measurement. Needle manipulation means 57 and septum-piercing needle 60 are for (i) piercing the septum of the container (sample vial 56) with the needle, (ii) withdrawing the first (test) substance from the container (sample vial 56) after the needle pierces the septum of the container and holding the withdrawn first substance in at least the passageway of the needle (see FIGS. 16 to 18), (iii) withdrawing the needle from the septum of the container and piercing the septum of the cuvette with the needle, and (iv) discharging the withdrawn first (test) substance from at least the passageway of the needle into the cuvette after the needle pierces the septum of the cuvette.

The needle manipulation means may be any structure (e.g., device) that can move, guide, and/or orient the septum-piercing needle and may include electronic circuitry and/or a programmable or programmed computer. The “septum-piercing needle” may be any structure capable of piercing the septum of each substance container (e.g., vials containing the substances in carrier fluid) and the septum of each testing chamber (e.g., cuvette) and withdrawing and expelling fluid.

In FIG. 1, needle manipulation means 57 comprises rail 59 and robotic arm 58, which holds septum-piercing needle 60 and optional needle guard 62, within which septum-piercing needle fits to protect the needle. Robotic arm 58 is movable laterally along rail 59 (left to right and right to left in FIG. 1) and carriage 61 can move along robotic arm 58 (approximately perpendicularly to the plane of FIG. 1), thereby allowing needle 60 to be moved above any of the sample vials, above any of the one or more cuvettes, above needle rinse chamber 48, and above waste port 50. Carriage 61 also has means for moving needle 60 up and down. Thus, needle 60 can be forced down to pierce the septum of any of the sample vials, cuvettes, etc. and it can be moved up so that it is withdrawn from the sample vials, cuvettes, etc. Robotic arm is desirably controlled by computer 46, preferably via a direct serial port connection.

Although not shown in FIG. 1, a needle guide is carried by robotic arm 58, and it serves at least two functions. It helps keep the needle from being deflected (bent) as it is forced down through the septa of the sample vials, the cuvette, etc. (although the exoskeleton of the septum-piercing needle usually provides more than enough structural rigidity), and, more importantly, the needle guide keeps each sample vial 56 from being lifted up out of sample vial racks 54 as the septum-piercing needle is pulled up out of the sample vial. The guide comprises a short hollow cylinder through which the needle passes, a short arm on which the cylinder is fixed, and a long rod parallel to and spaced from the straight longitudinal axis of the needle. The short arm is movable along the long rod so that the guide can be moved up and down along the rod and fixed at any location along with rod. Thus, the bottom of the guide can be semi-permanently fixed at a height just above the top of all of sample vials (which desirably are all of the same height). As the needle is being pulled upwards to withdraw it through the septum of a sample vial, if the vial is drawn upwards enough (because the septum of the vial is holding the needle so tightly that the needle is not being pulled free from the septum), the top of the sample vial will hit the bottom of the guide. Because the cylinder of the guide is fixedly mounted at the preset height (just above the top of the sample vial), the abutment of the sample bottle against the cylinder will prevent the bottle from drawn upward any further, thereby allowing the needle to pull free of the septum as the needle continues its upward movement.

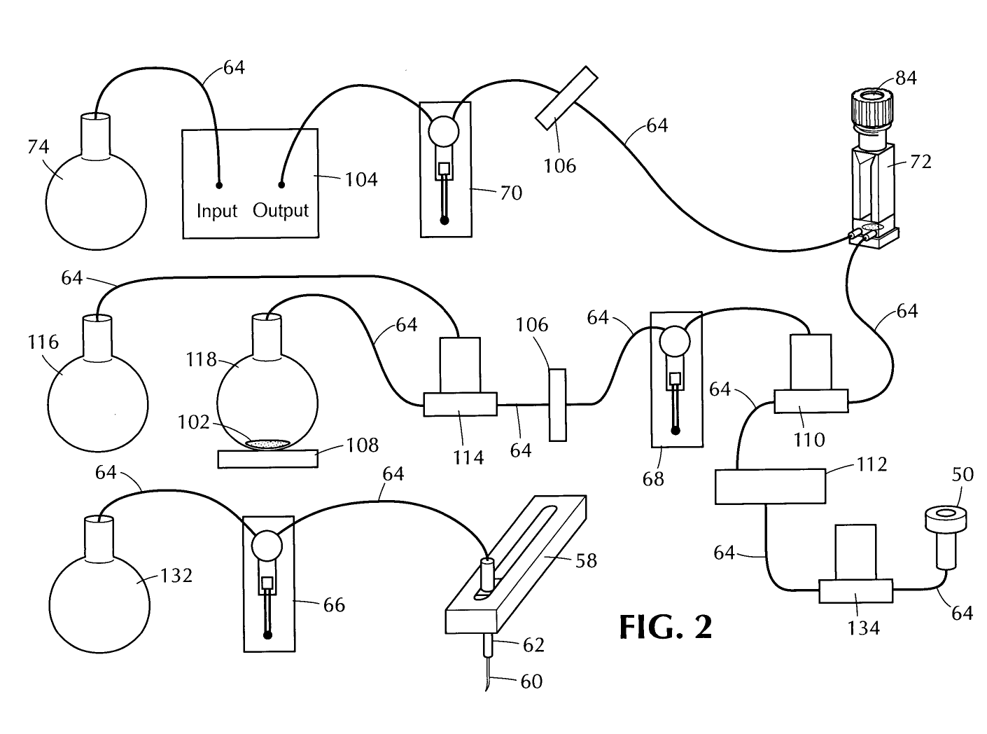

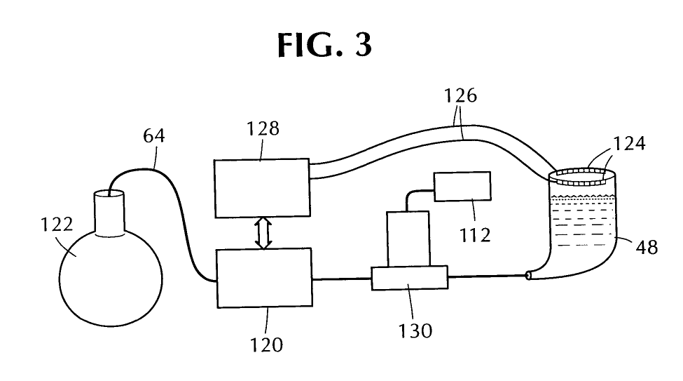

Tubing 64 connects needle 60 to first precision pump 66, which in turn is connected by tubing 64 to solvent reservoir 132 (not shown in this drawing; see FIG. 2). Second precision pump 68 and third precision pump 70 are also shown in FIG. 1. Each of precision pumps 66, 68, and 70 is connected by tubing 64 to cuvette and/or to various other fluid handling (e.g., pumps, valves) and/or storage components (solvent reservoirs, test fluid reservoirs) as shown schematically in FIGS. 2 and 3.

With reference now to FIGS. 2 and 3, which show the principle fluid handling and storage components, test fluid reservoir 74 holds the test fluid (e.g., a pH 7 chloride-free phosphate buffered solution) in which the kinetic solubility of the substance is to be determined. When third precision pump 70 is ordered (by the pump driver in the computer program, via the various cards and interfaces) to pump test fluid into cuvette 72, the test fluid is withdrawn from reservoir 74 through tubing 64, passes through degasser 104, through pump 70, through filter 106 (preferably a twenty micron or twenty-five micron filter), through more of tubing 64, and is pushed into cuvette 72, which has pierceable septum 84 and contains magnetic stir bar 102, which is driven by magnetic stirrer 108.

Solvent reservoir 116 holds a first solvent, preferably dimethyl sulfoxide, and solvent reservoir 118, which contains magnetic stir bar 102 driven by magnetic stirrer 108, holds a second solvent, preferably a cleaning agent that is mixture of “glass cleaner” (defined below) and ethylenediaminetetraacetic acid, which desirably is agitated by the magnetic stir bar to insure its homogeneity. This cleaning mixture is another aspect of the invention and is further described below. Three-way valve 114 allows either the first solvent from reservoir 116 or the second solvent from reservoir 118 to be drawn by second precision pump 68 through filter 106 (preferably a twenty micron or twenty-five micron filter) and pushed into one inlet port of three-way valve 110. An outlet of three-way valve 110 is connected to waste disposal 112. An inlet/outlet of three-way valve 110 is connected to the bottom of cuvette 72. Waste disposal 112 is also connected through two-way valve 134 to waste port 50, which is located on support surface 52 (see FIG. 1).

Solvent reservoir 132 holds a third solvent, preferably dimethyl sulfoxide. First precision pump 66 withdraws the third solvent from reservoir 132 and pumps the solvent through septum-piercing needle, which is held in robotic arm 58 by needle guard 62 (see also FIG. 1).

In FIG. 3, pump 120 under control of pump controller 128 draws solvent, preferably methanol (“MeOH”), from solvent reservoir 122 to fill needle rinse chamber 48 (see also FIG. 1). Desirably, one or more electrodes 124 are located at the rim of the needle rinse chamber to sense the solvent level within the needle rinse chamber and notify pump controller 128 via wiring 126 if the solvent level in rinse chamber 48 is too low. Two-way valve 130 is connected to the waste disposal 112 and allows the solvent in needle rinse chamber 48 to be withdrawn, preferably by vacuum, and sent to waste disposal 112.

The apparatus may be of any design and of any materials of construction that allow the benefits of this invention to be realized. The preferred Hach turbidimeter has already been mentioned; other parts of the apparatus are described below.

The preferred needle manipulation means comprises the Tecan RSP9000 Robotic Sample Processor, marketed by Tecan U.S., Inc. (Durham, N.C., United States, a subsidiary of Tecan Group AG, Mannedorf, Switzerland); however, other needle manipulation means can be used.

Any magnetic stir bar may be used. The preferred magnetic stir bar 102 is Model MSB-SX2 Magnetic Stir Bar, a slender and elongate iron rod about 6 millimeters long with a plastic coating, available from Starna Cells (Atascadero, Calif., United States). Any magnetic stirrer may be used. The preferred magnetic stirrer 108 is Model 9400 “Spinette” Cell Stirrer, also available from Starna Cells. Sonication or other appropriate mixing method may be used.

Precision pumps 66, 68, and 70 may be the same or different, depending on the fluids to be handled by the system. For screening drug candidates using the preferred kinetic solubility assay apparatus, Kloehn 48,000-step, syringe pumps (Model No. 50300), marketed by Kloehn Ltd. (Las Vegas, Nev., United States), are preferred. The volume of the syringe of pump 66 may be 50 microliters and the volume of the syringe of each of pumps 68 and 70 may be 5 milliliters. Because the Tecan RSP9000 Robotic Sample Processor unit is designed to be used with Cavro syringe pumps (which are marketed by Tecan U.S., Inc. but which are only 24,000-step syringe pumps) instead of Kloehn syringe pumps (which, as noted above, are 48,000-step), a new driver had to be installed to control the three Kloehn syringe pumps via an independent RS-485 (a standard communications protocol) card plugged into an ISA (a standard) slot on host computer 46. Kloehn-type syringe pumps are preferred in the present invention because they provide twice the precision as Cavro Syringe Pumps (48,000 steps v. 24,000 steps). Thus, the Kloehn syringe pump dispenses only half the amount of fluid per step (i.e., 0.5 microliters per step) as is dispensed by the Cavro syringe pump per step. Any pumps or other mechanisms for dispensing aliquots of liquid may be used, provided they are accurate enough, they can be controlled to the degree desired, their materials of construction are appropriate for the fluids being handled, the aliquots are small enough, etc.

Pump 120 is preferably a “Masterflex” Model 7543-60 (60 RPM) pump, available from Cole Parmer Instrument Company (Vernon Hills, Ill., United States), and pump controller 128 is designed for this service (the design is routine).

Electrodes 124, which sense the solvent level within the needle rinse chamber, comprise two copper braids commonly utilized to remove solder (by melting and then picking up the molten solder by capillary action), and are affixed to the rinse container using silicon adhesive.

Fluid reservoirs, whether for test fluid or solvents or other fluids, may be of any size, shape, or other design feature and constructed of any material that allows the intended functions to be performed properly (e.g., do not chemically interact with the fluids). Glass containers of 1800 milliliters have been found to be suitable.

The degasser may be of any size, shape, or other design feature and constructed of any material that allows the intended functions to be performed properly (e.g., do not chemically interact with the fluids). A preferred degasser is available from Orion Research Incorporated (England), Model No. RC3004.

The various filters may be of any size, shape, or other design feature and constructed of any material that allows the intended functions to be performed properly (e.g., do not chemically interact with the fluids). Preferred filters are available from Small Parts Co. (Miami Lakes, Fla., United States), Part No. 204060A, and are circular, one-half inch diameter, 20 micron, nylon filters.

The various valves may be of any size, shape, or other design feature and constructed of any material that allows the intended functions to be performed properly (e.g., do not chemically interact with the fluids). Preferred two-way and three-way valves are Part No. 4-213-900 (two-way) and Part No. 004-0053-900 (three-way) with Kalraz seats and seals, which are non-swelling when contacted by solvents, and preferred plug-in valve driver modules are Part No. 90-30-010-2, all of which are available from General Valve Division, Parker Hannifin Corporation (Fairfield, N.J., United States). Those valve driver modules are located on a custom valve drive board, which provides power to the individual drivers and whose design is readily apparent to one skilled in the art.

Needle rinse chamber 48 is a glass test tube having a curved nipple at the bottom, the major portion of which measures about 60 millimeters in height and 30 millimeters in diameter.

Waste port 50 is a nylon tube with a septum cap on one end (similar to that on the sample vials) and a hose barb at the other end (for connection to tubing).

Waste disposal 112 is a standard glass vacuum trap, a glass bottle with a plug at the top having two ports, one for the waste inflow and the other connecting to vacuum.

The design and material of construction of tubing 64 are not critical and may be any size for the expected flowrates, pressures, temperatures, and nature of the fluids to be transferred. The pieces of tubing connecting the various components may be the same or different. Typically for screening drug candidates for kinetic solubility, where small quantities are being handled, the tubing may be made of polytetrafluoroethylene, e.g., TEFLON® PTFE from DuPont (Wilmington, Del., United States), perfluorinated ethylene-propylene, e.g., TEFLON® FEP from DuPont, perfluoralkoxy fluorocarbon resin, e.g., TEFLON® PFA from DuPont, or ethylene tetrafluoroethylene, e.g., TEF-ZEL® from DuPont. In some cases, metal tubing (e.g., copper) may also be used. One skilled in the art will that recognize other homo-, co-, and terpolymers, plastics, rubbers, and mixtures thereof, as well as other metals, may be used. Preferred tubing measures 0.062 inches inner diameter and 0.125 inches outer diameter and is available from Upchurch Scientific, a division of Scivex (Oak Harbor, Wash., United States), Part No. 1521.

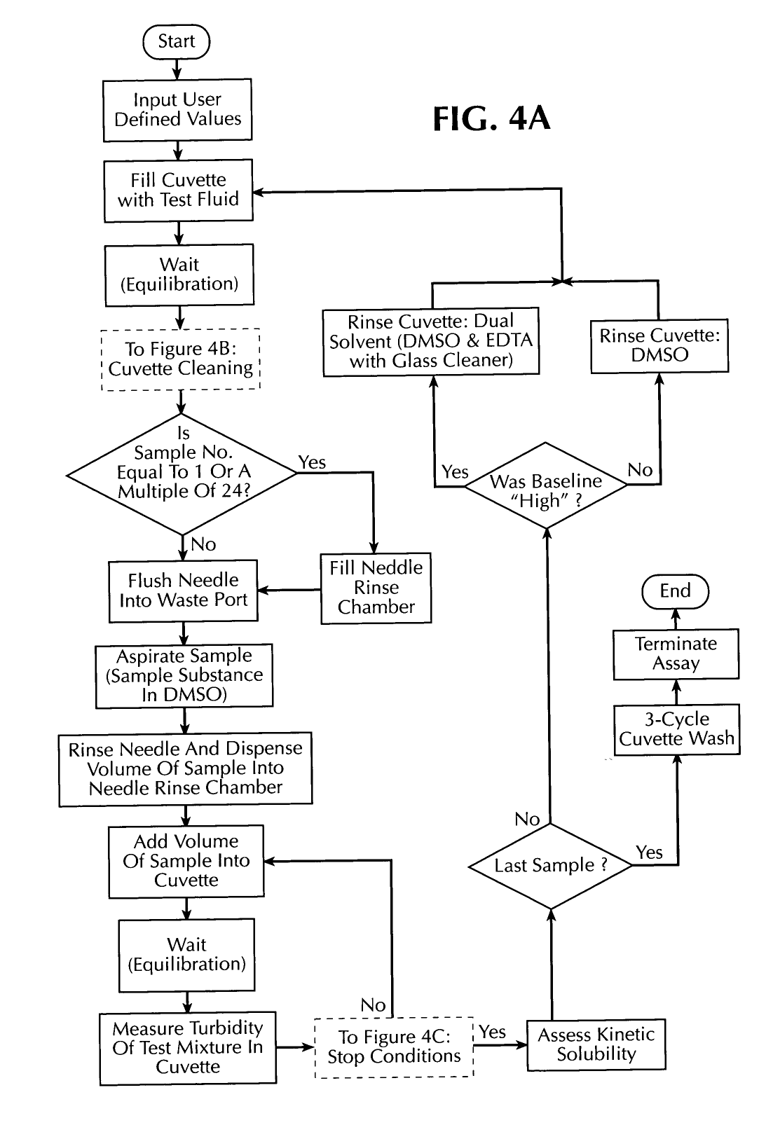

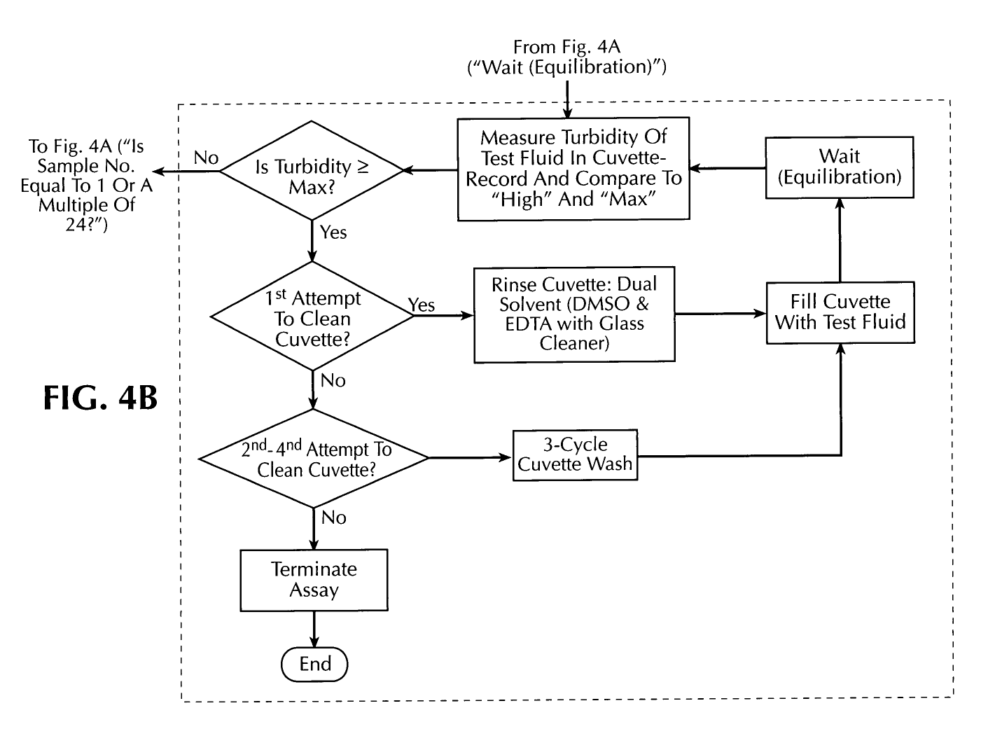

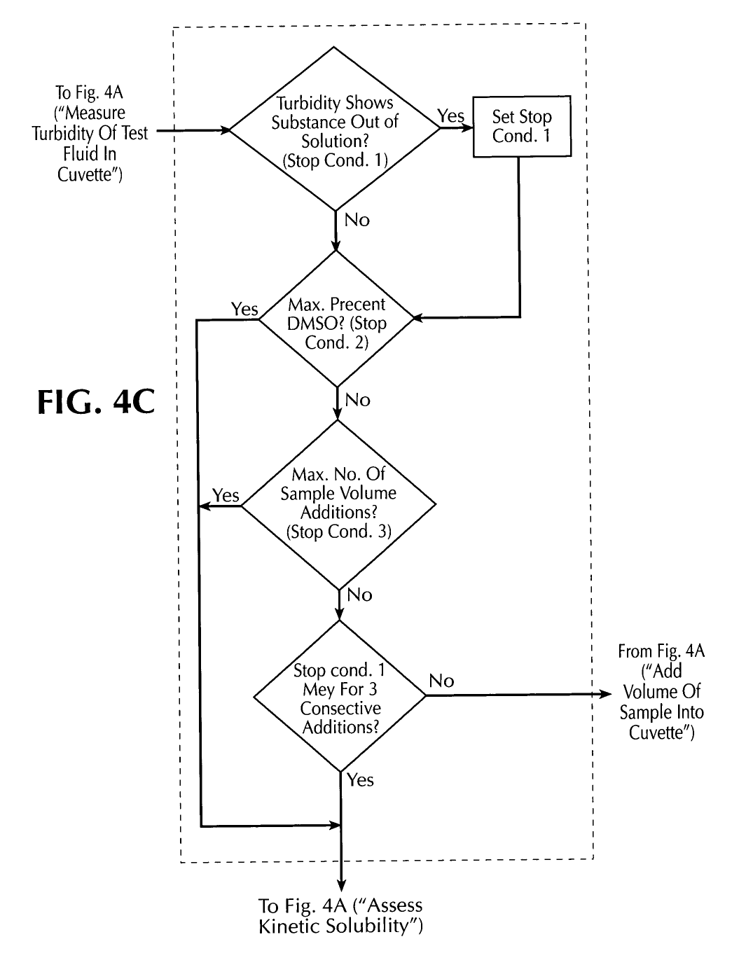

The block diagram of FIGS. 4A, 4B, and 4C show the principle steps of the preferred process performed by the kinetic solubility assay apparatus of FIG. 1. The block diagram assumes that fluid reservoirs 74, 116, 118, 122, and 132 of the apparatus have been filled with the appropriate fluids prior to beginning the assay and that there are one or more sample vials 56 in one or more sample vials racks 54. The block diagram also assumes that cuvette 72 (see, e.g., FIGS. 5 to 7) has been placed into special cuvette holder 136 (see, e.g., FIGS. 8 to 11), which is located within the body of the turbidimeter beneath sample port 36 in its normal position, i.e., so that pierceable septum 84 is accessible to septum-piercing needle 60 (see FIG. 1). For the sake of convenience, and because it is preferred, the discussion of the process of the block diagram also assumes that the assay is controlled by a computer running a process control program (e.g., the preferred program, whose source code listing is set forth herein). Finally, the discussion also assumes that the carrier liquid for the test substances is DMSO and that the test fluid is an aqueous liquid, e.g., a pH 7 chloride-free phosphate buffered aqueous solution (in other words, that the kinetic solubility determinations being made are of the test substances in an aqueous medium); however, as discussed herein, other carrier liquids (instead of DMSO) may be used (or possibly none used at all) and the test fluid need not be aqueous.

With reference now to the process block diagram in FIG. 4A, at the start of a series of assay runs, an operator may define the values for certain variables used by the computer's control software or, optionally, the operator may use a set of preprogrammed default values. Those values may include the number of sample vials 56 and their location in sample trays 54, the concentration of each test substance in the carrier fluid in the sample vials, the maximum allowable number of additions to the cuvette of each test substance before such additions are halted, the volume of test fluid to be placed in the cuvette initially for each test substance, and the high and maximum turbidity values (which are used in determining how and when the cuvette should be cleaned). Preferably, a graphical user interface (shown on a standard monitor connected to the computer) is provided to allow the operator to readily define those values.

Cuvette 72 is filled with test fluid (the pH 7 aqueous buffered solution) from test fluid reservoir 74 using precision pump 70 (see FIG. 2), which feeds the test fluid to the cuvette via one of the fluid ports 92, which desirably are at or near the bottom of the cuvette (see FIGS. 5 to 7). The test fluid is the cuvette may be agitated by any suitable means (e.g., sonication). Preferably the test fluid is agitated using magnetic stir bar 102 inside the cuvette, which is controlled by magnetic stirrer 108. Agitation may be performed for any desired length of time, but 30 seconds has been found to be suitable.

The test fluid in the cuvette is then allowed to equilibrate (i.e., rest undisturbed so that any entrained bubbles of air or other gas may escape from the fluid). Accuracy of turbidity measurement is improved if there are no air bubbles (or bubbles of any other gas) in the test fluid. The period for such equilibration may be of any length, but 90 seconds has been found to be sufficient. Too long a wait period reduces the number of samples that can be processed in a given amount of time, and long equilibration periods are not necessary with the liquids most often used in kinetic solubility assay (which typically have viscosities, densities, surface tensions, and other properties that allow substantially all of any entrained gas bubbles present to leave the liquids within no more than about 90 seconds). In the preferred cuvette, the gas bubbles leaving the liquid can exit the cuvette through vent 90, which is located above the maximum liquid fill line of the cuvette (see FIGS. 5 to 7).

After waiting the desired time for equilibration (FIG. 4A), the turbidity of the test fluid in the cuvette is measured using the turbidimeter (see first step in FIG. 4B: “Measure Turbidity . . . ”) The turbidity value of the test fluid in the cuvette prior to any addition of a test substance serves as a baseline measurement for determining which cuvette cleaning operations will be used during the assay. The baseline is classified in one of three categories: (i)<high (i.e., less than high), (ii)≧high <max (i.e., equal to or greater than high but less than maximum), and (iii)≧max (i.e., equal or greater than maximum). The turbidity baseline values are indicative of the relative cleanliness of the cuvette. If the measured turbidity equals or exceeds the “max” value (see decision diamond in FIG. 4B labeled “Is Turbidity≧Max?”), then the cuvette is considered to be too dirty to accurately measure the turbidity of a test mixture. The “high” value is a lower level of dirtiness that is low enough to allow the assay to continue; however, if the turbidity is equal to or above the “high” value but still below the “max” value, the cuvette will require more than just the usual (i.e., single solvent) rinsing prior to assaying the next substance to be tested. If the turbidity is less than “high,” the cuvette will require the least stringent cleaning. The “max” value depends on the particular apparatus being used and assay being run and can readily be determined (a preferred method for determining the “max” baseline value is discussed below). The “high” value may be set as a percentage of the “max” value and although it may be set at any level below “max,” one preferred value is 70% of the “max.”

If the measured turbidity does not exceeds the “max” value (in other words, in FIG. 4B, the question “Is Turbidity≧Max?” is answered “No”), the cuvette is considered to be sufficiently clean to proceed to the next step of the assay (see decision diamond in FIG. 4A labeled “Is the Sample No. a Multiple of 24?”). However, if the measured turbidity does exceed the allowable “max” baseline, an aggressive cuvette cleaning process is initiated (in other words, in FIG. 4B, the question “Is Turbidity≧Max?” is answered “Yes”).

On the off-chance that the greater-than-maximum baseline reading is anomalous (e.g., the reading is skewed because of an air bubble in the light path), the first attempt to clean the cuvette, which attempt is not as vigorous as any required subsequent cleaning steps (described below), entails a dual solvent rinse of the cuvette (see decision diamond in FIG. 4B labeled “1^{st} Attempt To Clean Cuvette?”). Preferably the first solvent is DMSO and the second solvent is a cleaning agent that is another aspect of the present invention (described below). Other acceptable solvents for cleaning the cuvette are methanol and acetone. Any solvent may be used provided it is a sufficiently effective cleaner and is otherwise acceptable (e.g., compatible with the materials of construction it contacts, etc.).

The cleaning agent of this invention comprises a mixture of ethylenediaminetetraacetic acid (“EDTA”) and “glass cleaner,” which itself is a mixture of water, ammonium hydroxide, 2-propanol, 2-butoxy ethanol, and anionic surfactant, preferably sodium dodecylbenzenesulfonate. It has surprisingly been found that such mixtures, preferably having a concentration of EDTA from 0.001% w to 50% w and more preferably from 0.01% w to 25% w, are especially effective at cleaning the cuvette. The increased effectiveness of these mixtures improves the accuracy of the assay and avoids wasting time and materials because of excess sample residue accumulation in the cuvette.

The dual solvent rinse proceeds as follows (see box in FIG. 4B labeled “Rinse Cuvette: Dual Solvent . . . ”). Test fluid in cuvette 72 is drained out of the cuvette through one of the two fluid ports 92 at the bottom of the cuvette (see FIGS. 5 to 7) and sent to waste disposal 112 through three-way valve 110 under suction supplied by pump 68 (see FIG. 2). The DMSO (the first solvent) in reservoir 116 is withdrawn by pump 68 through three-way valve 114 and filter 106 and pumped into the cuvette 72 via fluid port 92 through three-way valve 110, which has been reset so that fluid entering the valve goes to the cuvette and not to waste disposal 112. In similar fashion, the second solvent (the cleaning agent, which is the preferred mixture of EDTA and glass cleaner) is withdrawn from reservoir 118, and sent by pump 68 via a fluid port 92 into cuvette 72. The DMSO and cleaning agent, which are both present in the cuvette (and which together are sometimes referred to herein as a “cleaning mixture”), are agitated for about 30 seconds (by the magnetic stir bar) and are then drawn out by suction provided by pump 68 through three-way valve 110 (which has again been reset) and sent to waste disposal 112. The cuvette is then filled with test fluid from reservoir 74, agitated using the magnetic stir bar, and allowed to equilibrate (to allow any gas bubbles to leave the liquid). Another turbidity measurement is taken and if the turbidity does not exceed the “max” value, the cuvette is considered to be sufficiently clean and the assay continues to its next step (the decision diamond in FIG. 4A labeled “Is the Sample No. a Multiple of 24?”). If the cuvette is still too dirty (i.e., the turbidity value is above the “max”), the cuvette cleaning routine enters its second phase (in other words, in FIG. 4B, the question “Is Turbidity≧Max?” is answered “Yes” and the question “1^{st} Attempt To Clean Cuvette?” is answered “No”).

The second attempt to clean the cuvette is more aggressive and is referred to as a “3-cycle wash.” In this 3-cycle wash, two “cycles” (first and second separate washes) are performed with solvents and one “cycle” (a third separate wash) with test fluid.

For the first of the three cycles of the 3-cycle wash, the cuvette is emptied of test fluid in the manner previously described and then filled with a first solvent, preferably DMSO, from solvent reservoir 116 by pump 68. If desired, the speed with which pump 68 fills the cuvette can be adjusted to increase the force of the solvent flow into the cuvette (and thereby provide increased cleaning action). Pump 68 then draws all the solvent out of the cuvette 72 through one of the two ports 92 and then immediately pumps it back into the cuvette. This “in and out” pumping of the solvent may be performed as many times as desired; ten times is preferred. The solvent may be agitated by magnetic stir bar 102. After the final refilling of the cuvette with the first solvent, the solvent is agitated, preferably for at least 300 seconds. The solvent is then drained from the cuvette and sent to waste disposal 112 in the manner previously described.

For the second cycle of the 3-cycle wash, the cuvette is filled with a second solvent, preferably the cleaning agent that is a mixture of glass cleaner and EDTA (described above), which is drawn from solvent reservoir 118. As in the first cycle of the 3-cycle wash, the second solvent is pumped in and out of the cuvette as many times as is desired (preferably ten times and with increased force provided by pump 68 to provide increased cleaning action), and preferably agitated within the cuvette by magnetic stir bar 102. The second solvent is then drained and sent to the waste disposal 112.

For the third cycle of the 3-cycle wash, the cuvette is filled with the test fluid (e.g., the pH 7 chloride-free phosphate buffered aqueous solution), preferably from test fluid reservoir 74 by precision pump 70. As in the first and second cycles, the test fluid is pumped in and out of the cuvette for as many times as is desired (preferably ten times and with increased force provided by pump 70 to provide increased cleaning action). The test fluid is then drained from the cuvette and sent to waste disposal 112. Twice more the cuvette is filled with test fluid, pumped in and out of the cuvette, each for the desired number of times (with agitation), and sent to the waste disposal. The 3-cycle wash is then complete.

The cuvette is then filled again with test fluid from the test fluid reservoir, agitated, allowed to equilibrate (to allow any entrained gas bubbles to leave), and the turbidity is measured. If the turbidity does not exceed the “max” value, the cuvette is considered to be sufficiently clean and the assay continues to its next step. However, if the cuvette is not yet sufficiently clean (i.e., the turbidity is still above the “max” value), the 3-cycle wash is repeated, for up to a total of three times (see FIG. 4B). Those three 3-cycle washes plus the initial dual solvent rinse provide a maximum of four attempts to clean the cuvette if the turbidity exceeds the maximum allowable value. If upon the completion of the third 3-cycle wash the baseline turbidity in the cuvette still exceeds the “max” value, the apparatus shuts down and the assay is terminated (i.e., in FIG. 4B, the question “2^{nd}-4^{th} Attempt To Clean Cuvette?” is answered “No”). This premature termination of the series of runs (i.e., before the test substances in all of the sample vials have been assayed) avoids wasting substance samples, test fluids, solvents, and time and, more importantly, prevents inaccurate kinetic solubility assessments.

When the cuvette is sufficiently clean (i.e., when the question in FIG. 4B “Is Turbidity≧Max?” is answered “No”), the process moves to the next step of the assay (see decision diamond in FIG. 4A labeled “Is the Sample No. Equal to 1 or a Multiple of 24?”). At the start of a series of assay runs, the sample number (“Sample No.”) is set to a value of 1 and the answer to the question “Is the Sample No. Equal to 1 or a Multiple of 24?” will be “Yes,” at which point the process moves to the step of filling needle rinse chamber 48 with solvent (the reason for asking that question is explained below).

As shown in FIG. 3, pump 120 draws solvent, preferably methanol, from solvent reservoir 122 to fill needle rinse chamber 48. Preferably, one or more electrodes 124 are located at the rim of the needle rinse chamber to sense the solvent level in that chamber. Electrodes 124 are connected by wiring 126 to pump controller 128, which causes pump 120 to refill the needle rinse chamber with solvent if the electrodes signal that the solvent level is too low. Two-way valve 130 allows the solvent within the needle rinse chamber to be drained, preferably under suction, and sent to waste disposal 112. Because sample residue may accumulate in the needle rinse chamber after many samples have been assayed (i.e., needle 60 will have been rinsed many times, as described below), the apparatus can be set to drain needle rinse chamber 48 and then refill it with solvent after a certain number of samples have been assayed (after every 24 samples has been found to be satisfactory).

Simultaneously with the needle rinse chamber being filled (or optionally after it has been filled) or if the answer to the question “Is the Sample No. Equal to 1 or a Multiple of 24?” is “No,” needle manipulation means 57 positions septum-piercing needle 60 above waste port 50 with the end of needle 60 sufficiently below the upper end of waste port 50 so that liquid ejected from the end of needle 60 does not splash. Optionally, the top of waste port 50 is sealed with a septum, through which the lower end of needle 60 passes before any liquid is ejected from the needle. Needle 60 is flushed with solvent (preferably DMSO) withdrawn by pump 66 from reservoir 132, and the solvent leaving needle 60 is discharged into waste port (50). Preferably, three aliquots of 3.0 microliters each (a total of 9 microliters) are dispensed into waste port 50 in this manner. After the final discharge, needle 60 remains filled with the solvent (preferably DMSO).

Now, with needle rinse chamber 48 and needle 60 both filled with their appropriate respective solvents, needle manipulation means 57 positions needle 60 above the first sample vial 56. Septum-piercing needle 60 is forced downward through the septum in the top of the sample vial. It is another feature of this invention that the sample vials remain closed (thereby protecting the operator and preventing contamination) during the automated kinetic solubility assay performed by the apparatus of this invention. The preferred cuvette is described in further detail, e.g., in connection with FIGS. 5 to 7. Preferably the substance is present in the sample vial dissolved in DMSO because of DMSO's ability to dissolve a wide range of substances. Pump 66 pulls a volume of solution into the needle and associated apparatus that is at least as large as the total volume required to complete the assay even if the maximum allowable number of additions of solution of the test substance to the cuvette is made. That total amount can be approximated as the maximum number of sample volumes plus 1 to be added to the cuvette plus the volume to be used to “pack” or load the septum-piercing needle with the solution of the substance being assayed (see below). For example, when the maximum number of additions is set at 40, the addition volume is set at 0.5 microliters, and the packing volume is 0.2 microliters, the amount of sample aspirated will be at least 20.7 microliters (40 plus 1 equals 41, multiplied by 0.5 equals 20.5, plus 0.2 equals 20.7 microliters). (See box in FIG. 4A labeled “Aspirate Sample . . . . ”)

Sample-filled needle 60 is then withdrawn from sample vial 56 and dipped into needle rinse chamber 48 by needle manipulation means 57. With the tip of needle 60 submerged below the surface of the solvent in needle rinse chamber 48, an initial volume of sample, preferably 0.2 microliters, is dispensed into the solvent in order to pack the septum-piercing needle 60 with the solution of test substance to eliminate any air bubbles that may have entered the needle through its end and also to eliminate any solvent from the rinse chamber that may have entered the end of the needle. If an initial volume (e.g., 0.2 microliters) were not ejected prior to the needle's being put into the cuvette for delivery of sample solution, the first aliquot ejected from the needle into the cuvette (i.e., the first addition of test substance) might not contain a homogeneous solution of test substance in carrier fluid (in this case, DMSO) and would likely contain less (and possibly much less) of the test substance (because of the presence of the air and/or solvent from the rinse chamber), thereby introducing error into the kinetic solubility determination. (See box in FIG. 4A labeled “Rinse Needle And Dispense Volume Of Sample . . . . ”)

Needle 60 is then withdrawn from needle rinse chamber 48, repositioned by needle manipulation means 57 above sample port 36 in turbidimeter 32, and forced down through septum 84 in top 82 of cuvette 72 so that the end of the needle is immersed in the fluid already in the cuvette. A volume of sample, preferably 0.5 microliters, is dispensed into the test fluid within the cuvette, thereby forming a testable mixture, and agitated by magnetic stir bar 102, preferably for 30 seconds, to assure that the substance is adequately dispersed within the test mixture. During agitation, the testable mixture is partially drawn out of cuvette 72 by syringe 70 (the liquid level in the cuvette is not drawn down below the top of the magnetic stir bar) and pumped back into the cuvette to help insure good mixing of the newly added aliquot of test substance into the testable mixture.

The testable mixture is allowed to equilibrate, preferably for 90 seconds (i.e., to allow any entrained gas bubbles to leave the liquid), and the turbidity is measured (see boxes in FIG. 4A labeled “Wait . . . ” and “Measure Turbidity . . . ”). The process then determines whether one of the “stop conditions” for the assay is satisfied and, if so, the kinetic solubility of the sample is assessed and the assay continues to its next step (see box in FIG. 4A labeled “Assess Kinetic Solubility”). However, if none of the stop conditions are satisfied, the assay on that substance (as combined with the test fluid in the testable mixture) proceeds with further additions of test substance into the cuvette until one of the stop conditions is satisfied (see box in FIG. 4A labeled “Add Volume Of Sample Into Cuvette”). The stop conditions are based on the turbidity of the testable mixture, the percent of DMSO in the test mixture, and whether the user-specified maximum number of additions of sample solution to the cuvette has been made. The computer may be programmed to perform an additional number of additions of substance to the cuvette and turbidity measurements (preferably two) to verify that one or more of the stop conditions have in fact been satisfied.

Starting at the left side of FIG. 4C, the first stop condition is satisfied if the substance being tested has come out of solution. To determine if this has occurred, the turbidity of the current testable mixture (i.e., after the most recent sample addition) could be compared to the turbidity of the testable mixture prior to that addition. A sudden increase in turbidity would indicate that the most recent addition to the cuvette of the substance being assayed caused a suspension to form (i.e., the substance was longer completely dissolved in the test fluid in the cuvette). However, in the preferred assay, to determine whether the first stop condition obtains, the turbidity of the testable mixture is compared to the baseline value, which as described above, is the turbidity of the test fluid without any of the test substance being present. The turbidity of the testable mixture exceeding the baseline value by a predetermined amount indicates that the substance has formed a suspension in the testable mixture in the cuvette (i.e., that the substance of interest has come out of solution). The predetermined amount of increase is preferably equal to 3 times the standard deviation of the system.

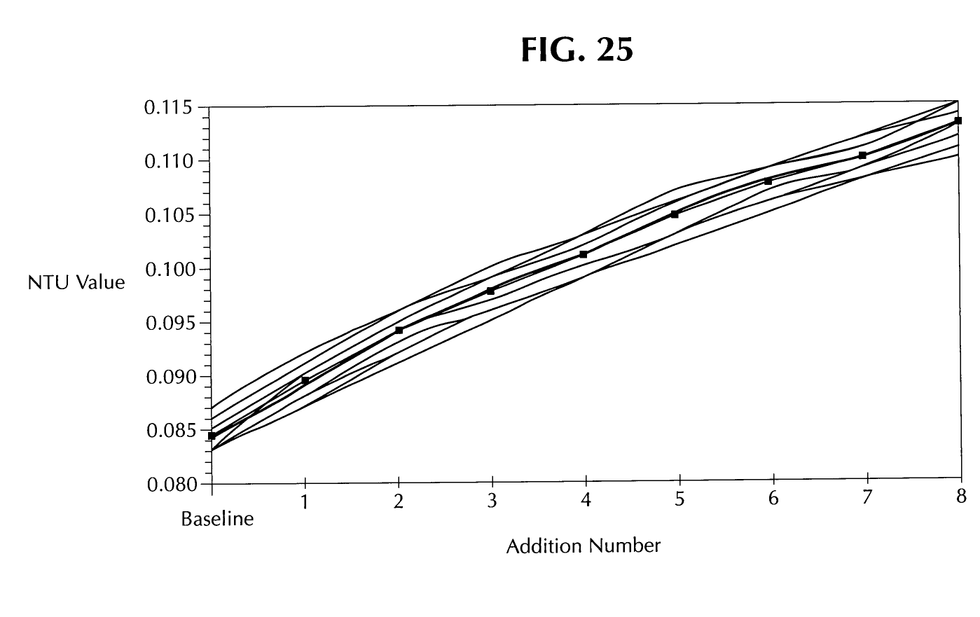

For example, assuming a pH 7 chloride-free phosphate buffered aqueous solution is used as the test fluid with the Hach 2100N turbidimeter modified in accordance with the present invention, after performing a statistically significant number of turbidity measurements following additions of a standard solution (e.g., a 1000 NTU standard) to the test fluid in a clean cuvette (which set of experiments is discussed below in connection with FIG. 25), the mean average standard deviation might be determined to be 0.001 NTU (the mean average standard deviation would be the simple mean average of the standard deviations, one standard deviation having been determined for each set of replicate data points at each given number of additions). Such experiments also yield the mean average baseline turbidity of the test fluid in the cuvette (i.e., prior to addition of any standard solution). Thus, if the mean average baseline turbidity is 0.080 NTU for the test fluid prior to any addition of the substance whose kinetic solubility is to be determined, a turbidity reading for a testable mixture that exceeds 0.083 NTU (0.080, plus 3 multiplied by 0.001) would indicate that the test substance was no longer in solution. Whether or not the first stop condition is satisfied, the second stop condition is examined (in FIG. 4A, the decision diamond labeled “Max. Percent DMSO?”) and the “yes-no” (true-false) status of the first condition is stored for later use (the reason for this is explained below).

Because substances whose kinetic solubilities are being determined using the apparatus and assay of this invention are typically dissolved in DMSO, the second stop condition is met if the testable mixture contains more than a maximum allowable amount of DMSO (which maximum allowable amount can be specified by the operator). DMSO concentration in the testable mixture in the cuvette must be monitored when determining kinetic solubilities in aqueous media because it is known in the art that excessive amounts of DMSO tend to increase the solubility of a compound in an aqueous medium. If the test fluids are not aqueous media, monitoring DMSO (or other carrier liquid) concentration may not be necessary (although the DMSO or other carrier liquid might have an adverse effect on whatever non-aqueous test fluid is being used).

The maximum percentage of DMSO in the testable mixture is preferably lower than 5% per volume, more preferably lower than 1% v/v, and most preferably lower than 0.65% v/v. The DMSO concentration in the test mixture is computed based on the total volume of fluid within the cuvette, the amount of DMSO in the sample solution containing the substance being tested, and the amount of sample solution added to the cuvette. For example, if the sample solution has a concentration of 10 micrograms of substance per microliter of sample solution (which means that for calculation purposes, the sample solution may be assumed to be substantially all DMSO), the cuvette contains 2 milliliters (2000 microliters) of test fluid, the sample additions are made in volumes of 0.5 microliters to the testable mixture, and the maximum allowable DMSO of the total test mixture is set at 0.65% v/v, then the maximum allowable DMSO concentration will be reached by the twenty-sixth addition of sample solution to the cuvette (i.e., 26 additions of 0.5 microliters equals 13 microliters of DMSO, and those 13 microliters added to 2000 microliters of test fluid results in a concentration of 13 microliters of DMSO divided by 2013 total microliters of testable liquid mixture, which equals 0.65% v/v). If the second stop condition is met (i.e., the maximum allowable concentration of DMSO has been reached), the kinetic solubility is assessed. If the second stop condition is not met, the third stop condition is considered.

The third stop condition is met when the maximum allowable number of sample solution additions to the cuvette has been made. The maximum allowable number of additions is a user-defined value and may be designated at the start of the series of assay runs. A preferred default value is 40 additions. If the third stop condition is met, the assay proceeds to assess the kinetic solubility of the substance (see decision diamond in FIG. 4C labeled “Max. No. Of Sample Volume Additions?”). If the third stop condition is not met, a final condition is examined.

The final condition is met if the first stop condition (i.e., turbidity increase greater than a specified amount) is satisfied after each of three consecutive sample additions to the testable mixture (see decision diamond in FIG. 4C labeled “Stop Cond. 1 Met For Three Consecutive Additions?”). Requiring this final condition helps assure that one or even two anomalous turbidity readings (e.g., because of entrained air bubbles) do not prematurely end the assay. If the final condition is not met, another sample volume is added to the test cuvette, followed by equilibration, turbidity measurement, etc. (see box in FIG. 4A labeled “Add Volume Of Sample Into Cuvette?” and the boxes following it). If the final condition is met, the assay proceeds to assess the kinetic solubility of the substance.

If the first stop condition has been satisfied for three consecutive additions of sample volume (0.5 microliters), i.e., the final stop condition has been satisfied (and the second and third stop conditions have not been satisfied), the kinetic solubility is assessed based on the turbidity reading taken after the first addition that caused the first stop condition to be met. Thus, for example, if the turbidity for the testable mixture indicates that the substance is not in solution for each of three consecutive sample solution additions, the kinetic solubility is assessed based on the turbidity reading taken after the first of those three additions, not the turbidity readings taken after the second or third additions.

If the second stop condition is satisfied (and the first and third stop conditions have not been satisfied), the kinetic solubility of the substance is based upon the concentration at the time the maximum allowable DMSO content is reached. For instance, using a maximum allowable DMSO concentration of 0.65% v/v, a sample solution with a concentration of 10 micrograms of test substance per microliter of sample solution, a cuvette holding 2 milliliters (2000 microliters) of test fluid, and sample solution additions made in 0.5 microliter increments, the kinetic solubility reported at the maximum allowable DMSO concentration will be 65 micrograms of test substance per microliter of testable mixture. Twenty-six additions each of 0.5 microliters of sample solution (which is the maximum number of allowable additions; see above discussion of the second stop condition) containing 10 micrograms of test substance per microliter of sample solution gives 130 micrograms of substance added, divided by 2.013 milliliters (i.e., 2013 microliters, which is 26 multiplied by 0.5, plus 2000) equals 65 micrograms per milliliter.

If the third stop condition is met (and the first and second stop conditions have not been met), the kinetic solubility of the substance in the test fluid is reported as being equal to or greater than the number of micrograms of substance per milliliter of test mixture at the time the maximum number of sample volume additions has been made. For example, assuming a maximum of 40 additions of sample solution allowed, the sample solution having a concentration of 10 micrograms of test substance per microliter of DMSO, a cuvette volume containing 2.0 milliliters (2000 microliters) of test fluid, sample additions made in 0.5 microliter increments, the kinetic solubility reported at the maximum number of additions would be≧99 μg/ml, which is calculated as follows. Forty additions of sample solution multiplied by 0.5 microliters per addition multiplied by 10 micrograms of test substance per microliter of DMSO equals 200 micrograms of substance added to the cuvette, the liquid in the cuvette has a volume of 2.02 milliliters (40 additions multiplied by 0.5 microliters per addition, plus 2000 microliters, equals 2020 microliters), and 200 divided by 2.02 equals 99 micrograms per milliliter.

Regardless of which stop condition causes the kinetic solubility of the substance then under consideration to be assessed, the kinetic solubility and some or all of the underlying data may be stored in memory and/or exported to another program for further analysis.

With reference now to the decision diamond in FIG. 4A labeled “Last Sample?,” once the kinetic solubility of the most recent substance being processed has been assessed, the assay compares the total number of samples already processed against the total number of samples to be processed to determine if there are any additional samples to be processed.

If no more samples are to be processed, the assay is terminated in the following manner. Needle 60 is withdrawn from cuvette 72 and all fluid is flushed from needle 60 into waste port 50. Needle 60 is rinsed in needle rinse chamber 48 and returned to a starting position (e.g., the position shown in FIG. 1). Needle rinse chamber 48 is drained and the drainage is sent to waste disposal 112. Cuvette 72 is given the 3-cycle wash described above, after which all fluid is drained from it and sent to waste disposal 112. Apparatus 30 is then partially or completely powered down and the series of assay runs is considered to have been terminated.

If additional samples are to be assayed, needle 60 is withdrawn from cuvette 72 and the baseline turbidity reading for the sample run just completed is examined to see if it was “high.” If it was not, cuvette 72 is rinsed with DMSO from solvent reservoir 116 in the manner described above. If the baseline for the sample run just completed was “high,” the cuvette is given the dual solvent rinse described above (i.e., with DMSO from solvent reservoir 116 and the mixture of glass cleaner and EDTA from solvent reservoir 118). In either case, cuvette 72 is filled with test fluid from the test fluid reservoir 74 and the assay continues as previously described until there are no more samples to be assayed (see box in FIG. 4A labeled “Fill Cuvette With Test Fluid”).

With reference to FIGS. 5 to 7, preferred cuvette 72 has bottom 80, top 82, and wall 86 between them and connected to both, the three of which together define enclosed volumetric space 88 for receiving fluid (including the test fluid and the substance whose kinetic solubility in the test fluid is to be determined). Pierceable septum 84, which forms part of the top, allows fluid to be injected through it into the volumetric space within the cuvette. Vent 90, a through-hole preferably 1.0 millimeters in diameter and near the top of the cuvette, allows gas to flow in and out of volumetric space 88, e.g., as the volume of liquid in the cuvette changes. The gas will usually be air, unless, for example, the cuvette is being used under a gas pad, e.g., in a laboratory enclosure padded with nitrogen. Cuvette 72 typically has an internal volume for holding liquid (i.e., the volume below vent 90) of from 0.3 milliliters to 5 milliliters and preferably 2 milliliters. One or more fluid ports 92, preferably located at or near the bottom of the cuvette, allow adding liquid to and removing liquid from the volumetric space of the cuvette. Tubing 64 is connected to ports 92 by fluid port connectors 93, which may be screw connectors or couplings that can pushed onto the nipples of ports 92 and which are held in that position by friction fit. FIG. 6 shows the cuvette of FIG. 5 containing a small amount of liquid 81 in the lower region of the cuvette. FIG. 7 shows the cuvette of FIG. 5 almost completely filled with liquid 81. As will be understood by one skilled in the art, the volumetric space inside the cuvette need not be of uniform cross-section. Thus, for example, in the lower region of the cuvette, the volumetric space has a larger cross-sectional area (e.g., square), which provides sufficient room for magnetic stir bar 102 to rotate (see FIG. 5), while the volumetric space above the lower region is more rectangular in cross-section, e.g., to provide a thinner layer of testable mixture for the test (interrogation) light of the turbidimeter to pass through (compare FIGS. 6 and 7).

Screw cap 96 has internal threads (not shown), which mesh with threads 94 near the top of the cuvette for holding screw cap 96 tightly against the rest of the cuvette (with pierceable septum 84 in between) when cap 96 is screwed onto threads 94, thereby sealing the top of the cuvette and helping to safeguard the operator from contact with the contents of the cuvette (the test liquid, the substances being tested, and the one or more carrier liquids). Cap 96 has centrally located opening 98 to allow the substance transfer means, preferably a septum-piercing needle, to penetrate septum 84 when inserted in the direction indicated by arrow 100. Pierceable septum 84 may be made of any suitable material but will typically be polymeric, e.g., one or more rubbers, silicones, plastics, and combinations thereof. Septum 84 may be made of the same material as used for tubing 64 and/or for the septum at the top of each sample vial 56. The septum is desirably self-sealing after being punctured.

Cuvette 72 may be made of any material that is pervious to the energy being used to measure the turbidity of the test fluid or test mixture within the cuvette. Preferred materials include, but not limited to, quartz, glass, transparent and rigid plastics, and any other sufficiently transparent and rigid materials known to those of skill in the art.

A preferred quartz cuvette is a square lateral cross-section 2.0-milliliter volume cuvette from Starna Cells, Inc., Atascadero, Calif. The outer dimensions of the cuvette, not including the cylindrical portion having vent hole 90 and threads 94 (which is preferably screwthread GL 14), are 12.5 millimeters by 12.5 millimeters by 60.5 millimeters high. The internal dimensions of the lower region of the cuvette where the magnetic stir bar is located are 10 millimeters by 10 millimeters by 5.0 millimeters high. Above that region, the internal dimensions below chamfered surfaces 89 in upper region (just below vent 90) are 4.0 millimeters wide by 10.0 millimeters thick by 43 millimeters high, the narrow width being desirable to allow the interrogation light of the turbidimeter to pass through. The two ports 92 each have an internal diameter of 1 millimeter. The body of the cuvette is quartz. The septum is silicone rubber with a plastic backing.

FIGS. 8 to 11 show preferred cuvette holder 136, which has the shape of a right cylinder (preferably about 25.4 millimeters in diameter, about 87.3 millimeter high, with a wall thickness of 1.5 millimeters) and which may be used to hold a cuvette of the present invention, particularly the preferred cuvette of FIGS. 5 to 7. During operation, cuvette holder 136 is normally placed into the turbidimeter through sample port 36, cuvette 72 is placed into cuvette holder 136, and both are held by retainer 37 in the cavity of which sample port 36 is the entryway (see FIG. 1). The bottom of cuvette 72 rests on ledge 137 of the cuvette holder.

Light inlet 138, through which the incident (or interrogation) light from the turbidimeter enters the cuvette, is a through-hole whose center is about 31.75 millimeters from the bottom and which measures about 7.94 millimeters in diameter. Rear light outlet 140 is a race-track shaped through-passageway, formed by two semi-circles located at opposite ends of the outlet and each having a radius of about 3.175 millimeters. The center of the lower semi-circle is about 31.75 millimeters from the bottom of the cuvette holder and the centers of the two semi-circles are about 15.88 millimeters apart. Side light outlet 142 is a race-track shaped through-passageway, formed by two semi-circles located at opposite ends of the outlet and each having a radius of about 1.588 millimeters. The center of the lower semi-circle is about 34.93 millimeters from the bottom of the cuvette holder and the centers of the two semi-circles are about 12.7 millimeters apart. Rear light outlet 140 is 180 degrees from forward light inlet 138, and side light outlet 142 is half-way between them (i.e., is at a 90 degree angle to each of inlet 138 and outlet 140). Light inlet 138 is narrow to reduce the amount of scattered light produced, and light outlets 140 and 142 are narrow to reduce the amount of light scattered by the cuvette's square corners that can reach the two light detectors of the turbidity means (i.e., at the side and rear), thus eliminating a source of error. Desirably, any scattering detected is desirably caused only by the presence of particles in the testable mixture in the cuvette and not by the cuvette itself; however, a small cuvette will itself cause some scattering. Hence, the desirability of reducing the amount of detected non-particulate scattering (i.e., the scattering caused by something other than particulates) to the extent possible.

Side opening 144, defined at its bottom by ledge 137 and at its two sides by parallel longitudinal edges 145 (which edges are 12.7 millimeter apart), provides clearance for the nipples of ports 92 of cuvette 72 but is only slightly wider than the outermost surfaces of the two ports 92 (FIG. 11). Accordingly, when cuvette 72 is slid into cuvette holder 136, the cuvette is properly oriented within the cuvette holder and cannot be in any other rotational orientation with respect to the cuvette holder (see FIG. 11), which itself has been fixed in the cavity below sample port 36 of the turbidimeter at the proper height and in the proper rotational orientation with respect to the light source and the two light detectors of the turbidimeter. All of this insures that the four planar portions of the wall of the cuvette are perpendicular to the three small and precisely located wall openings through which light is introduced (i.e., forward light inlet 138) and allowed to exit (i.e., rear light outlet 140 and side light outlet 142), which in turn reduces errors in the turbidity measurements taken. In FIG. 11, circular wall 146 of the cuvette holder surrounds the top of screw cap 96, and the central portion of pierceable septum 84 is visible through opening 98 in the top of screw cap 96.

Cuvette holder 136 may be made of any suitable material (e.g., metal, rigid plastic, wood, fiberglass) and may be 1.0 to 2.0 millimeters thick.

The principal function of the cuvette holder is to support the cuvette in the energy (e.g., filtered light) path provided by the turbidimeter. The cuvette holder may have any shape and size that holds the cuvette and does not interfere with or adversely affect the turbidity measurements (e.g., allows the light or other energy source appropriate access to the cuvette, allows the light or other energy beams leaving the cuvette appropriate access to the one or more detectors of the turbidimeter, and does not otherwise introduce any significant errors into the turbidity measurement). If the cuvette to be used is of the size and shape normally used in the turbidimeter, a cuvette holder in addition to any normally present in the turbidimeter may not be necessary. In the preferred apparatus of this invention, the additional cuvette holder (i.e., cuvette holder 136) is employed because the preferred cuvette is smaller than the testing chamber (e.g., test tube or cuvette) normally used with the preferred (Hach 2100N) turbidimeter.

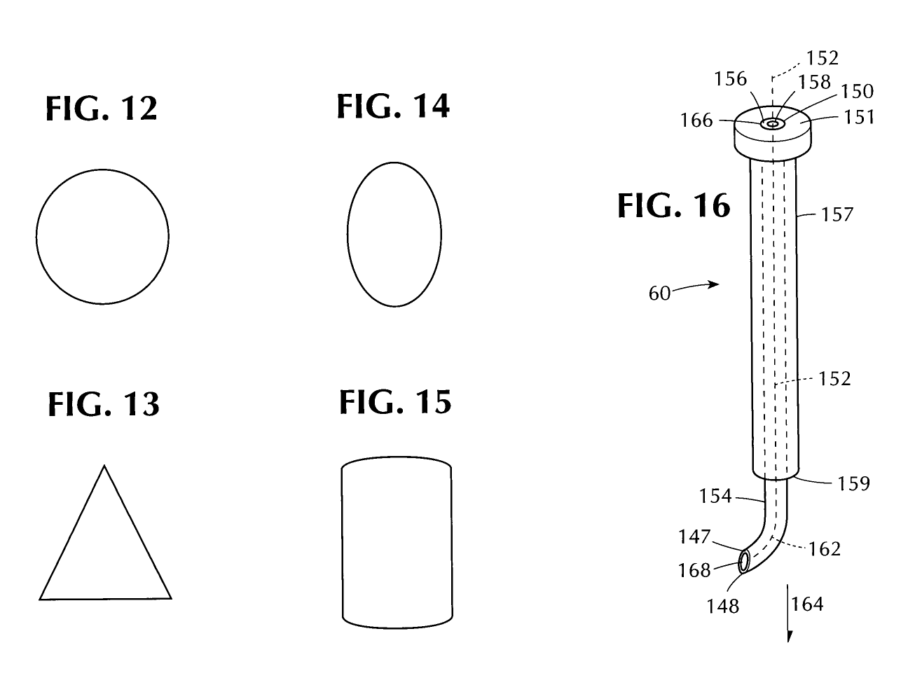

Similarly, the cuvette may have any design, shape, and size that holds the amount of testable mixture being employed, allows the appropriate turbidity measurements to be made, and allows the kinetic solubility assay apparatus to be automated. The preferred cuvette has a square lateral cross-section. FIGS. 12 to 15 show alternative lateral cross-sections that could be employed with other cuvette holders and possibly with other turbidimeters: circular (FIG. 12); triangular (FIG. 13), which just like a rectangular lateral cross-section has at lest three planar surfaces; curved (FIG. 14 as well as FIG. 12); and any shape having at least four surfaces, at least two of which are planar and are parallel to each other (e.g., FIG. 15). The cross-sections shown are not intended to be limiting and any other shape known to those of skill in the art may be used as long as the benefits of the present invention can still be achieved.

Another aspect of the invention is the septum-piercing needle, which is desirably used in the kinetic solubility assay apparatus of this invention for passing through the septa of sample vials 56 to remove samples (e.g., solutions of the compounds to be tested dissolved in DMSO) and for passing through the septum of the cuvette to add aliquots of samples to the test fluids (e.g., pH-buffered solutions) in the cuvette. Preferred septum-piercing needle 60, shown in FIGS. 16 to 18, has upper straight section 153, lower curved section 155, piercing end 147 (which has lower piercing point 148 and oppositely disposed upper point 149), non-piercing end 150 (which is preferably fixed within annular member 151), longitudinal axis 152 (including curved portion 162, which lies within and corresponds to lower curved section 155 of the needle), and outer sleeve 157 (for the sake of clarity, shown only in FIG. 16), with a tilted lower edge whose lowest point is indicated by reference numeral 159. Lower curved section 155 of needle 60 is typically from about 1% to 10%, desirably from about 2% to about 8%, and preferably from about 3% to about 6%, of the overall length of the needle. In a preferred embodiment, lower curved section 155 is 4 millimeters long and upper straight section 153 is 75 millimeters to 100 millimeters long. Rigid exoskeleton 154 of needle 60 contains and supports corrosion-resistant cannula 156, which has central elongate passageway 158 (for holding fluid) comprising straight portion 160 (which corresponds to upper straight needle section 153) and curved portion 161 (which corresponds to lower curved needle section 155 and curved portion 162 of longitudinal axis 152). Angle 163 subtended by curved section 155 of the needle will typically be from about 80 degrees to about 100 degrees, but angles outside that range may be used in some cases.

Passageway 158 of cannula 156 preferably has an average diameter of from 100 to 300 microns and a length (measured along the longitudinal axis from the plane of non-piercing end 150 to plane 170 of piercing end 147) of from 10 to 150 millimeters, although diameters and lengths outside those ranges may be useful in certain cases. With a diameter of 300 microns and a length of 150 millimeters, passageway 158 would have a volume of approximately 10.6 microliters. With a preferred passageway diameter of 252 microns and a preferred passageway length of 95 millimeters, passageway 158 has a volume of approximately 4.73 microliters. As discussed above, the amount of fluid (containing test substance and any carrier fluid) removed from a sample vial must be at least about 20.7 microliters if a maximum of 40 aliquot additions of 0.5 microliters could be made, but the passageway of the preferred needle holds only a fraction of that fluid volume. Accordingly, it is apparent that what cannot be held in the passageway of the needle must be held in tubing 64 connecting the needle and syringe pump 66. Desirably, none of this fluid (containing the test substance) reaches the syringe of syringe pump 66.

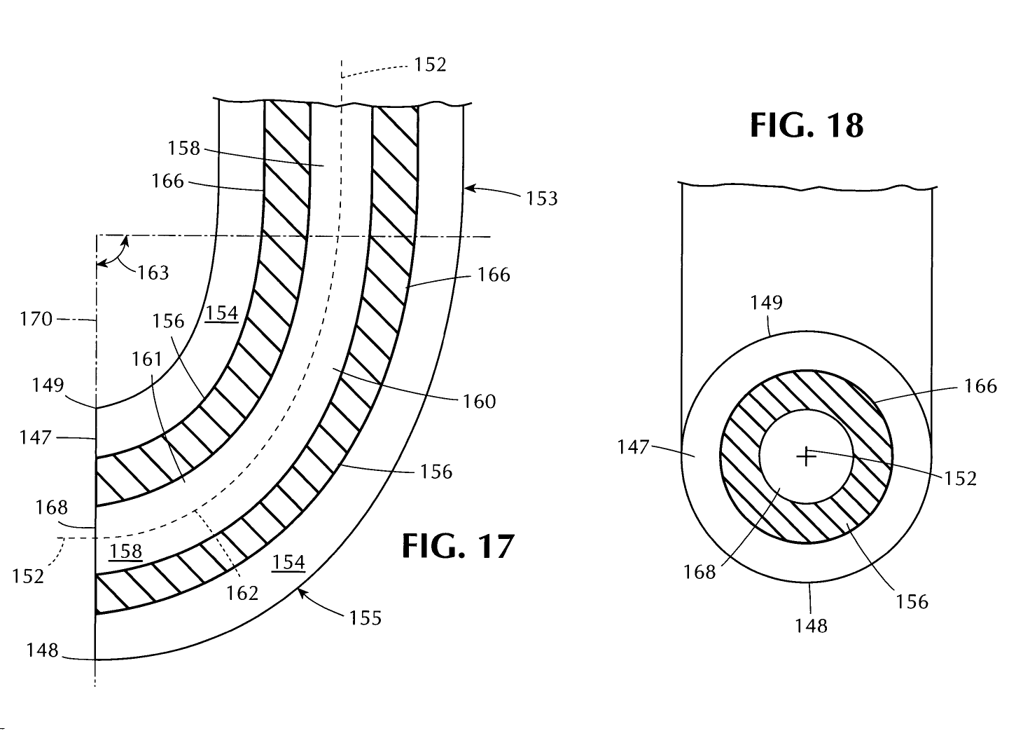

Piercing point 148, which lies in plane 170 and which, when needle 60 is oriented as shown in FIGS. 16 to 18, is the lowest point of piercing end 147 (and the lowest point of needle 60), can penetrate the septum of the cuvette or sample vial when inserted in the direction indicated by arrow 164 (which is the same direction indicated by arrow 100 in FIG. 5). Passageway 158 terminates at its lower end in opening 168, which lies in plane 170, and plane 170 is preferably parallel to the (upper) portion of longitudinal axis 152 in straight portion 160 of the needle, as shown in FIG. 17. Thus, piercing point 148 and upper point 149, both of which lie in plane 170, preferably lie in the direction of travel of the needle (arrow 100 in FIG. 5 and arrow 164 in FIG. 16) when it is being moved down and up (i.e., inserted into or removed from the sample vials and the cuvette) and, therefore, bottom opening 168 of passageway 158 faces left in FIG. 17.

With reference to FIG. 17, if the needle is constructed so that plane 170 is rotated clockwise while holding piercing point 148 immobile, bottom opening 168 of passageway 158 faces partially upward and if plane 170 is instead rotated counterclockwise, bottom opening 168 faces partially downward. Too much rotation in either direction is undesirable because that needlessly increases the width of the hole made by the needle and increases the possibility that opening 168 will become blocked by small pieces of the septum. For example, if opening 168 faces partially upwards (resulting from clockwise rotation of plane 170), small pieces of a septum may become embedded in the opening as the needle is being withdrawn from a sample vial or cuvette, and if opening 168 faces partially downwards (resulting from counterclockwise rotation), microscopic pieces of a septum may become lodged in the opening as the needle is being inserted into a sample vial or cuvette. In some cases, e.g., because of fabrication methods, it may be acceptable for plane 170 to be rotated clockwise as much as 15 degrees without the functioning of the needle and apparatus being adversely affected.

As shown in FIG. 16, outer shell 157 is straight and its lower extent terminates above the start of the curved section of the exoskeleton. The lowest point of the outer shell is indicated by reference numeral 159. That lowest point provides a natural place for any excess fluid to drip from the needle as the needle is moved up out of fluid.

Exoskeleton 154 may be made from any material that is sufficiently inert to the various fluids to which it is exposed, that provides sufficient rigidity so that the needle can pierce the septa of the sample vials and cuvette without suffering structural damage (e.g., permanent deformation), that can be worked satisfactorily to form the exoskeleton, and that can be satisfactorily bonded to the cannula. Metal is preferred and particularly preferred is stainless steel (e.g., the stainless steel used for medical syringe needles, scandium, titanium). Cannula 156 may be made from any material that is sufficiently inert with respect to the various fluids to which it is exposed, that can be satisfactorily bonded to the exoskeleton, and that has sufficient structural strength. Glass is preferred and particularly polymicro fused silica capillaries. Any method may be used to secure the cannula inside the exoskeleton that does not interfere with the integrity and functioning of the septum-piercing needle and adhesive is preferred (e.g., acrylate-type adhesives, Super Glue, epoxy, silicone). Reference numeral 166 in FIGS. 17 and 18 indicates the layer of adhesive between (outer) exoskeleton 154 and the (inner) cannula 156. Exoskeleton 154 preferably has outer shell 157 around it to provide additional structural rigidity.

The preferred cannula may be obtained from Polymicro Technologies, Inc. (Phoenix, Ariz., United States). The preferred inner diameter is 252 microns, although smaller or larger diameters may be used, and the preferred outer diameter is 357 microns. The preferred exoskeleton is of 304 stainless steel, 22 gauge, and six inches long and may be obtained from Popper and Sons, Inc. (New Hyde Park, N.Y., United States) with the required small curve at the bottom end. The preferred outer shell is also stainless steel and is obtained from Gilson, Inc. (Middleton, Wis., United States), Part No. 27067236. Depending on the inner diameter of the cannula, the needle may be used for aspirating (withdrawing) and/or delivering (expelling) sub-microliter volumes, e.g., nanoliter volumes.

To make the septum-piercing needle, adhesive (preferably Super Glue) is applied to the exterior of the cannula and before the adhesive sets, the piece of cannula is inserted into the exoskeleton so that the end of the cannula extends beyond the end of the exoskeleton. After the adhesive sets, the curved end is trimmed to remove the cannula that protrudes beyond the end of the metal. That also prevents blockage of the cannula by adhesive, because any adhesive that has entered the cannula will be present in the section of cannula removed. The exoskeleton (with the cannula) is then slid into the outer shell, which is close fitting enough to provide a friction fit.

It has surprisingly been found that not only can the septum-piercing needle of this invention repeatedly accurately deliver small quantities of fluid (coefficient of variation in volume is less than 5% from aliquot to aliquot) and help safeguard operators and others (e.g., by allowing use of sealed containers both for transporting and testing the substances), but that the needle eliminates one of the possible sources of error encountered with the non-piercing glass needle used in the earlier assay. As noted above, in that earlier assay, small quantities of the various substances being tested built up outside the end of the needle to form hard deposits, and it is believed that these deposits in some cases adversely affect the solubility determinations. Although the reason is not understood, it has been found that such deposits do not build up on the septum-piercing needle of this invention.

FIG. 19 is a block diagram showing the main cards, interfaces, and computer that provide for automatic control of the turbidimeter of the apparatus of FIG. 1. One important feature of the apparatus of this invention is that it is “automated,” which, as indicated above, refers to the fact that the apparatus, under normal operating conditions and once it is stocked or supplied with reservoirs or sources of the one or more test fluids and other fluids used, a source of the one or more test substances (e.g., in containers such as small vials), etc., and is properly programmed and set (e.g., told how many sample vials it is to process), is capable of carrying out the intended process to completion (i.e., the determination of the kinetic solubility of all of the test substances) without the need for human intervention after the process has been initiated.

With reference to both FIGS. 1 and 19, computer 46 (e.g., a Compaq DESK PRO EN) is connected to and controls needle manipulation means 57 via standard serial connection (RS-232) to the turbidimeter and Tecan RSP 9000 and via RS-485 serial connection to the syringe pumps, using a card from Black Box Corporation (Lawrence, Pa., United States), Dual Port RS23214221485 Serial Interface, Part No. IC11 3C. Standard 50-pin bus ribbon cable 44a connects computer 46 (FIG. 1) via ISA bus interface from Keithley Instruments, Inc. (Cleveland, Ohio, United States), Part No. MID-64 Metra Bus Optical Isolated Interface Card, to three standard cards (also available from Keithley) in signal interface system 42 (FIG. 1). The first, an 8-channel relay card (Keithley MEM-08), is a high-current relay card for controlling higher amperage users in the system, e.g., turning on and off the pumps, valves, etc. The second, a 32-channel relay card (Keithley MEM-32), is a low-current relay card for controlling lower amperage users in the system, e.g., the Hach 2100N turbidimeter controls, and for resetting the peak detectors on the custom turbidimeter interface board (described below). The relays connected to the turbidimeter allow the computer program to complete (or close) the control circuits of the turbidimeter and thereby simulate a human operator pressing the buttons/touchpads of those turbidimeter control circuits (the completion or closing of those circuits is what occurs when an operator presses those buttons/touchpads). The third card, a 32-channel digital I/O (input/output) card (Keithley MBB-32; “MBB” indicates Metrabyte Metrabus), is a digital input and output card that reads signals from the custom turbidimeter interface card (which signals are either zero volts or five volts) and the rinse level detection circuit. The custom turbidimeter interface board is connected to the Hach 2100N turbidimeter by standard 50-pin bus ribbon cable 44b and provides pathways between the 32-channel I/O card and the MEM-32 relay card and the Hach 2100N turbidimeter. As shown in FIG. 19, signal interface system 42 comprises the three Keithley electronic interface cards (i.e., Keithley MEM-08, MEM-32, and MBB-32) and the custom turbidimeter interface board. The four boards (the three Keithley standard boards and the custom turbidimeter interface board) need not be located near each other and one or more of the cards may be in other locations. For example, the custom turbidimeter interface board may be located inside the turbidimeter itself and signals from it may available via a bus pin connector mounted in the housing of the turbidimeter.

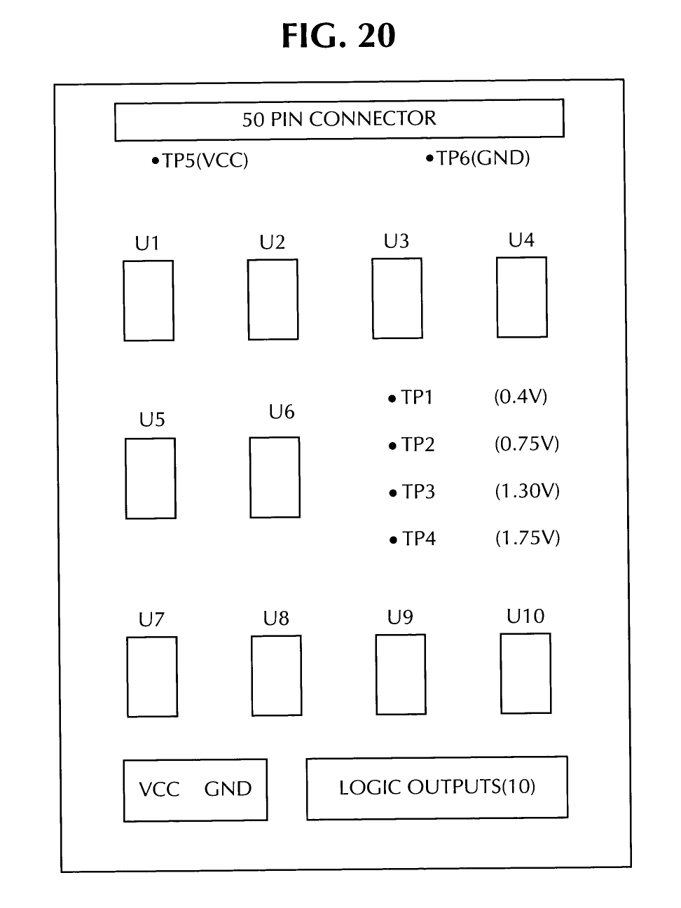

FIG. 20 shows the layout of the custom turbidimeter interface board. Fifty-pin connector cable 44b, running from the turbidimeter, connects to a 50-pin connector on the custom turbidimeter interface board, which is shown as a rectangle at the top of FIG. 20. The custom turbidimeter interface board contains ten similar integrated circuits (U1 through U10), each of which decodes information concerning one of ten important states of the turbidimeter, thereby allowing the computer to “know” what the turbidimeter's settings are at each moment of operation. “VCC” is the connector for system power (“SYSPWR”) to the board, and “GND” is the custom turbidimeter interface board's connector to ground. “TP” indicates a test point on the board. TP1 through TP4 have their respective test voltages shown next to them in parentheses (those voltages are also the trip points); TP5 is the test point for the supply voltage to the board; and TP6 is the test point for ground. A 10-pin connector makes available to the Keithley 32-channel digital I/O card (which is also on signal interface system 42) the ten logic outputs from the ten integrated circuits (U1 to U10).

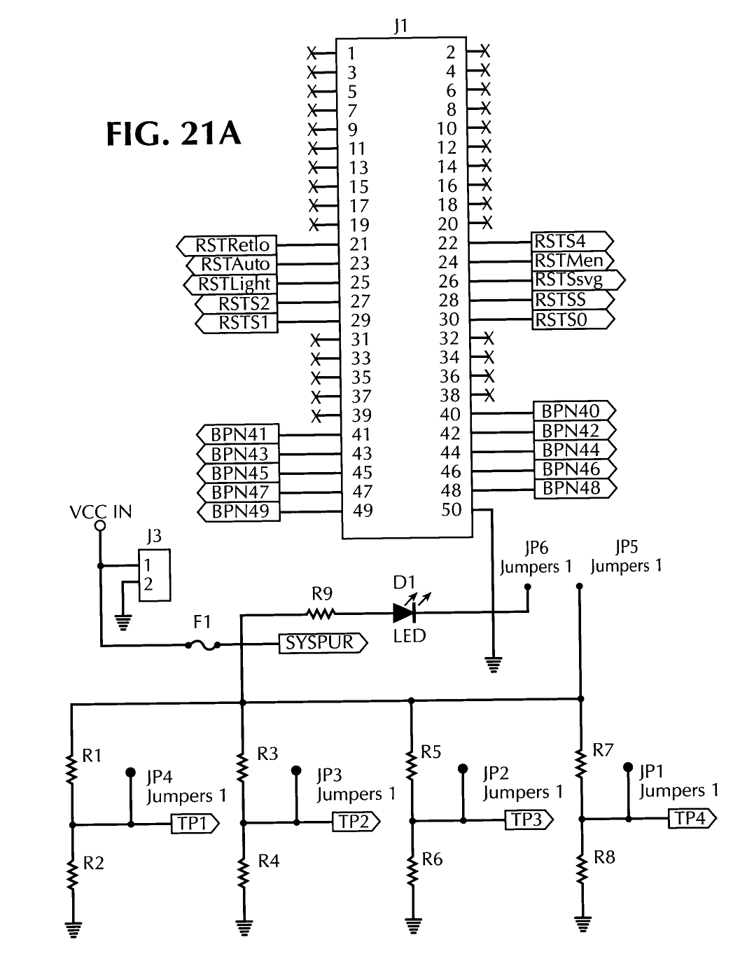

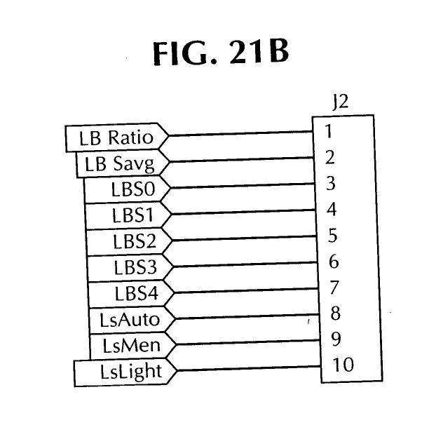

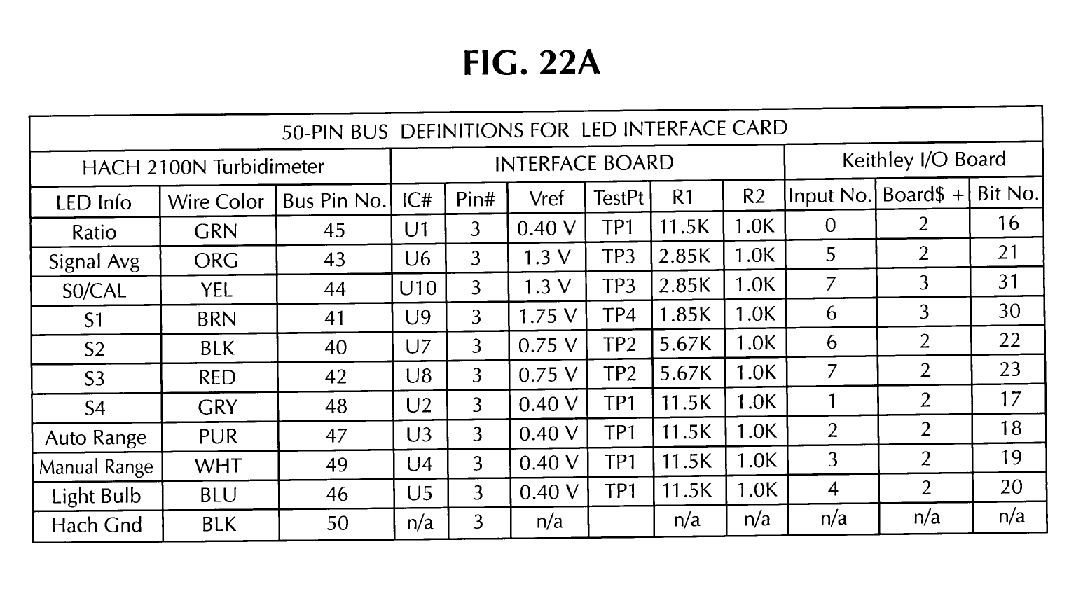

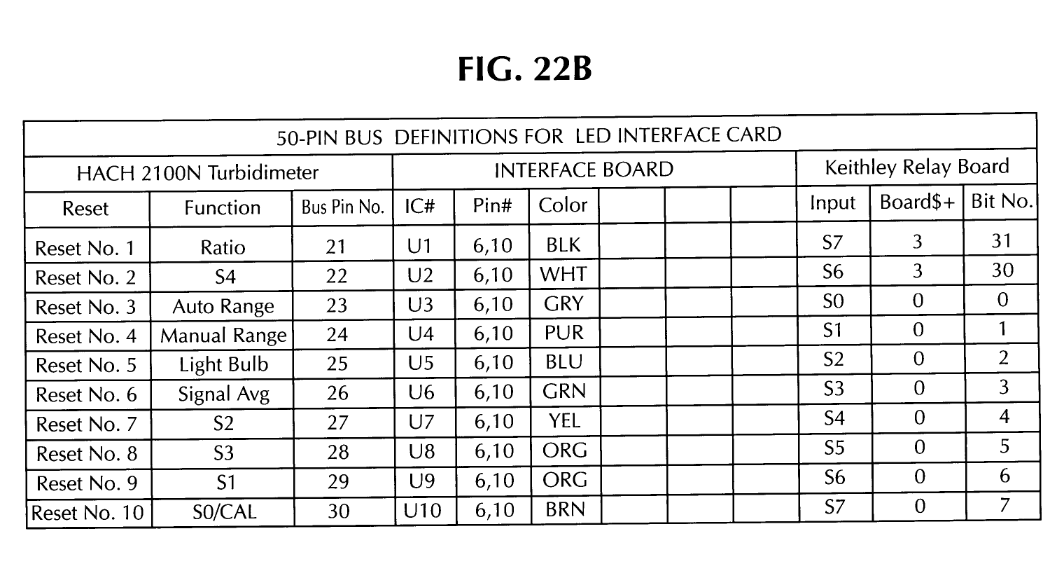

FIGS. 21A and 21B depict some of the circuitry and connectors on the custom turbidimeter interface board. J1 indicates the 50-pin connector (at the top of FIG. 20), which receives and sends signals to the turbidimeter, J2 indicates the 10-pin logic output connector (lower right corner of FIG. 20), the output signals from which are sent to the Keithley 32-channel digital I/O card, and J3 indicates the connector for power and ground (lower left corner of FIG. 20). FIGS. 22A and 22B show the correspondence between signals/functions of the turbidimeter, bus pins on J1, the integrated circuits (U1 to U10), and the Keithley I/O and 32-channel relay board.

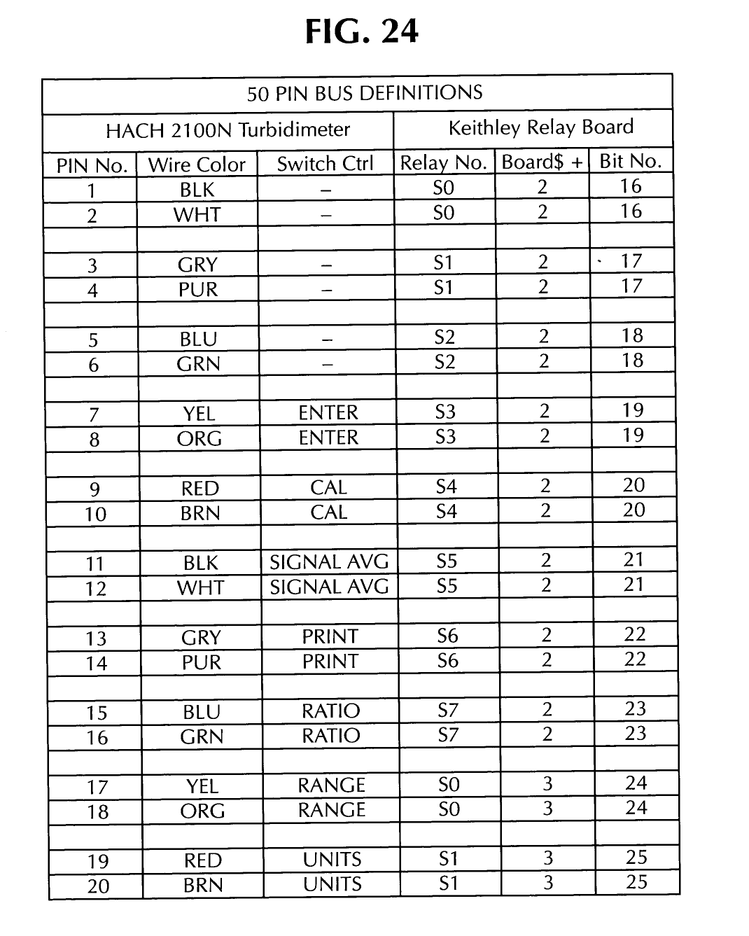

As shown in FIG. 21A, pins 1 to 20 are used to close contacts on the touch sensor buttons on the turbidimeter (assignments are shown in FIG. 24) and 31 to 39 on J1 are not used, and pin 50 goes to ground. The touch sensors are closed for 0.5 seconds using the MEM-32 relay board to simulate a keypress by an operator. Pins 40 to 49 of J1 receive information from the turbidimeter via jumpers attached to various turbidimeter LED (light-emitting diode) displays and 50-pin bus ribbon cable 44b, the information is processed by the ten integrated circuits on the custom turbidimeter interface board (U1 to U10) from an analog format to a digital format, the results are sent via connector J2 on the custom turbidimeter interface board to the Keithley I/O board, and the Keithley I/O board then reads the digital information and sends that digital information to the computer. The computer analyzes the information and sends signals to the various equipment, namely, relays on either of the two Keithley relay boards, the needle manipulation means, or back to the turbidimeter via the Keithley I/O card and the custom turbidimeter interface board. Signals sent by the computer to the turbidimeter pass through pins 1 to 30 on the bus connecting to cable 44b on the custom turbidimeter interface board and are used for relay contact closure and reset signals.

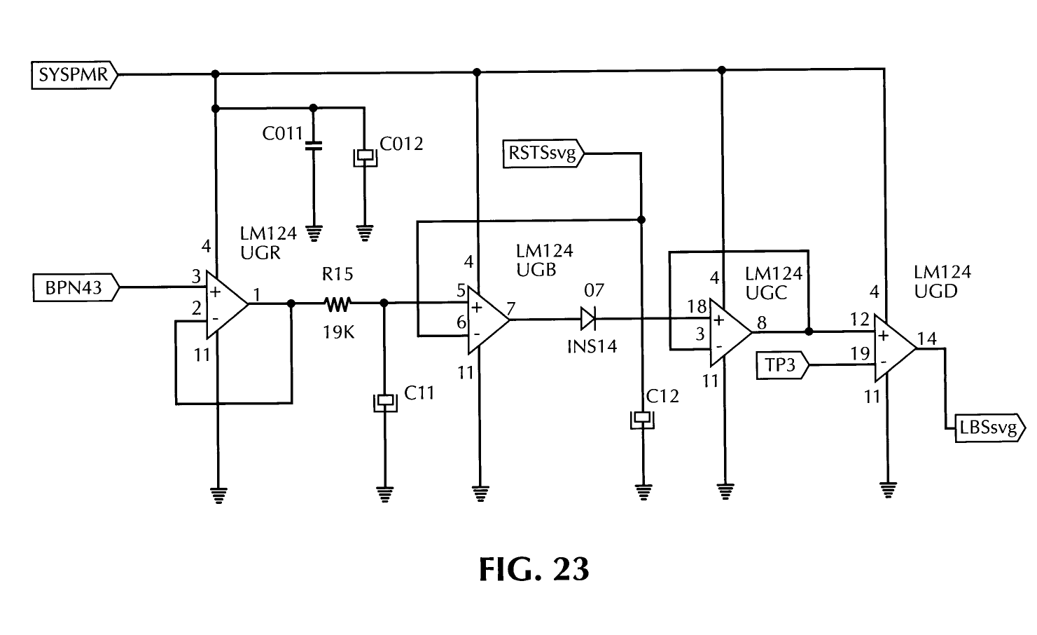

As show in FIG. 22A, information from the various LED displays on the Hach 2100N turbidimeter passes through each of bus pins 41 to 49 on the turbidimeter and pin 50 on the turbidimeter is used for ground. The same pin numbers are used on J1 of the custom turbidimeter interface board. Taking the row for “Signal Avg” as an example, and with reference also to FIG. 23, which is the schematic for a representative integrated circuit (in this case, U6), the color of the ungrounded jumper wire from the respective LED is “Org,” which indicates orange, and the signal passes through pin 43 on the turbidimeter (note that all LED connections are taken from the negative terminal on the turbidimeter except for the SO LED, which is taken from pin 4 on U2 or the negative terminal on LED S1). That signal is carried by 50-pin ribbon cable 44b and received by pin 43 on the custom turbidimeter interface board (“BPN43”), from which it flows to pin 3 of integrated circuit U6. The corresponding reference voltage is 1.3 volts, which appears as test point 3 (“TP3”; also see FIG. 20). R1 and R2 are used to create a reference divider for the appropriate reference voltage. The output of integrated circuit U6 (“LBSavg”) is available on pin 2 of J2 on the custom turbidimeter interface board (see FIG. 21B) and flows to the Keithley 32-channel I/O board (input number 5, on board address 2 ($ indicates address), bit number 21), from which it is sent through cable connector 44a to computer 46 (FIGS. 1 and 19).

If the computer determines that reset is necessary, the appropriate signal (“RSTSavg”) is grounded by a command sent back through cable connector 44a to the Keithley relay board MEM-32, which connects the input to be reset to ground (see FIG. 22B), and from there to the custom turbidimeter interface board through pin 26 of J1 (FIG. 21A; FIG. 22B, row entitled “Reset No. 6”), where it is fed to integrated circuit U6 (FIG. 23).

FIG. 24 sets forth the correspondence between the switch controls on the turbidimeter and the 32-channel relay card (board). When a button on the turbidimeter is to be “pushed” as a result of a signal being sent by the computer, the appropriate relay on the 32-channel relay board is closed.

Referring back to FIG. 23, as will appreciated by one skilled in the art, R15 (resistor R15 and capacitor C11 comprise a simple single-pole low-pass filter, which is used to capture the DC (direct current) response of the signal from the turbidimeter's circuitry. This type of simple filter is classified as single pole because the degree of the denominator polynomial of its transfer function is 1. The cutoff frequency of such filters is found using the following formula:

f_{cutoff}=1/(2πRC)

The operational amplifiers (the right-pointing triangles in FIG. 23 with two inputs and one output) are used to shift signals either to 0 to 5 volts, which are then read by the custom turbidimeter interface board as being either “on” (5 volts) or “off” (0 volts). When the input voltage to the comparator (the right-most operational amplifier) exceeds the reference voltage (TP3), the output voltage swings to the “high” rail or VCC. When the input voltage is below the reference voltage, the output voltage swings to the “low” rail or ground. In FIG. 23, there are four such operational amplifiers, the right-most one of which is utilized as a comparator to switch LBSavg between 0 volts and 5 volts.

The circuit shown in FIG. 23 also contains a peak detector, which stores the value of the input voltage in a capacitor until the capacitor is reset by discharging it to ground. That is done by means of a connection to a relay located on the custom turbidimeter interface board and controlled by the Keithley 32-channel relay card. At the appropriate time, the computer sends a signal to the 32-channel relay card to cause the relay to close, which then discharges the capacitor, thereby causing the desired reset. In FIG. 23, the peak detector comprises the second operational amplifier from the left (of the total of four operational amplifiers), diode D7, and capacitor C12.

Some of the abbreviations, names, and symbols used in FIGS. 21, 22, 23, and 24 are listed below and have the meanings indicated.

| ABBREVIATION/NAME/ | |
|---|---|
| SYMBOL | MEANING/COMMENT |
| IC # | Integrated circuit number (i.e., U1 to |
| | U10) |
| Pin # | Pin number |
| Vref | Reference voltage and the voltage at |
| | which the comparator trips |
| TestPt | Test point |
| R1 in FIG. 22A | Voltage divider Resistor 1 |
| R2 in FIG. 22A | Voltage divider Resistor 2 |
| Board$ + Bit No. | Address |
| RSTxxx | Reset; indicates reset of the value for xxx |
| | (“RSTSavg” indicates a reset of Savg) |
| BPNyyy | Bus pin number yyy (“BPN43” indicates |
| | bus pin number 43) |
| LBzzz | Output of comparator that goes to |
| | interface Logic Board |
| Ratio | Ratio button on Hach turbidimeter |
| Signal Avg | Signal average button on Hach |
| | turbidimeter |
| S0/CAL | S0/CAL button on Hach turbidimeter |
| S1 | S1 button on Hach turbidimeter |
| S2 | S2 button on Hach turbidimeter |
| S3 | S3 button on Hach turbidimeter |
| S4 | S4 button on Hach turbidimeter |
| Auto Range | Auto range LED on Hach turbidimeter |
| Manual Range | Manual range LED on Hach turbidimeter |
| Light Bulb | Light bulb LED on Hach turbidimeter |
| Hach Gnd | DC ground on Hach turbidimeter |
| Switch Ctrl | Function on turbidimeter being controlled |
| Up-pointing arrow (↑) | Up button on Hach turbidimeter |
| in FIG. 24 | |
| Right-pointing arrow (→) | Right button on Hach turbidimeter |
| in FIG. 24 | |
| Down-pointing arrow (↓) | Down button on Hach turbidimeter |
| in FIG. 24 | |
| Enter in FIG. 24 | Enter button on Hach turbidimeter |
| Cal in FIG. 24 | Cal button on Hach turbidimeter |
| Print in FIG. 24 | Print button on Hach turbidimeter |
| Range in FIG. 24 | Range button on Hach turbidimeter |
| Units in FIG. 24 | Units button on Hach turbidimeter |
| Rxx in FIG. 23 | Resistor number xx (e.g., in FIG. 23, R15 |
| | is resistor 15); resistances are in ohms |
| | (e.g., the resistance of R15 is 10,000 ohms) |
| Cyy in FIG. 23 | Capacitor number yy (e.g., in FIG. 23, |
| | C11 is capacitor 11); capacitances are in |
| | microfarads (e.g., the capacitance of C11 |
| | is 4.7 microfarads) |
| Dzz in FIG. 23 | Diode number zz (e.g., D7 is diode 7) |
| Right-pointing | Comparators, which have a high and a |
| triangles in FIG. 23 | low input and an output (e.g., the signal |
| | from bus pin 43 from J1 on the custom |
| | turbidimeter interface board is fed to pin |
| | 3 of the left-most comparator in FIG. 23) |
| GRN | Green |
| ORG | Orange |
| YEL | Yellow |
| BRN | Brown |
| BLK | Black |
| RED | Red |
| GRY | Gray |
| PUR | Purple |
| WHT | White |
| BLU | Blue |
| n/a | Not applicable |

One skilled in the art will recognize that the circuitry employed will depend on a number of factors, including the turbidimeter used, the characteristics of the one or more measuring chambers (e.g., cuvettes) used, the type of pumps, relays, etc. used, and the fluids to be moved (e.g., how many cleaning solutions are used), and will also recognize that even for the preferred apparatus described, many different circuits could be used. One skilled in the art will have no trouble designing circuitry for a particular kinetic solubility assay and the apparatus chosen to implement it.

As indicated above, as far as is known to applicant, the Hach 2100N turbidimeter (just like other commercially available turbidimeters) is designed for measuring the turbidity of waste, sewage, and other streams typically having turbidities orders of magnitude higher than the turbidities to be encountered in the kinetic solubility assay of this invention and the Hach 2100N turbidimeter is designed to use a measuring chamber (test tube or cuvette) significantly different in cross-section and larger than the preferred cuvette used in the modified turbidimeter. Accordingly, it was necessary to calibrate the preferred apparatus of this invention (which includes the square lateral cross-section 2 milliliter cuvette, the preferred cuvette holder, etc.) so that it would be sufficiently sensitive and precise when used for kinetic solubility assays. In other words, it was necessary to recalibrate the turbidimeter to be more sensitive in the range in which the system was to be used. Furthermore, it was also necessary to determine for the modified turbidimeter the relationship between the apparent turbidity displayed on the LED and the actual turbidity because the turbidity signal taken from the turbidimeter (i.e., the signal displayed on the turbidimeter's LED) for further processing (by the computer program written by applicant) is produced by the unmodified internal circuitry of the turbidimeter. One skilled in the art will recognize that such calibration will be necessary for each particular apparatus to be used.

The specifications of the Hach 2100N turbidimeter when used with its standard circular lateral cross-section cuvette or test tube, which has a volume significantly larger than the 2.0 milliliter volume of the preferred square lateral cross-section cuvette, are as follows:

| Characteristic | Hach Display (NTU) |
|---|---|
| Resolution | 0.001 |
| Repeatability | Greater of ±0.01 or 1% |

Resolution is the smallest change in turbidity that can be determined and displayed by the turbidimeter. In this case, repeatability is indicated by the size of the range of turbidity readings that are determined and displayed for each of one or more given samples. The smaller the value (for the unmodified Hach 2100N unit, the greater of 0.01 or 1%), the smaller the range (scatter) of possible second, third, fourth, etc. readings that are obtained for a given sample (in other words, the smaller the value, the greater the likelihood that the same turbidity value will be determined and displayed each time a given sample is re-assayed). Repeatability is usually determined by assaying a statistically valid number of different samples each a statistically valid number of times, where the samples have accurately known turbidities (e.g., provided by standards) throughout the working range expected to be encountered when assaying unknowns. With a repeatability of the greater of 0.01 NTU or 1% (as for the unmodified Hach 2100N turbidimeter), a sample that has a known turbidity of 50 NTU would be expected to produce readings in the range of 49.5 NTU to 50.5 NTU (i.e., 50 minus 1%, to 50 plus 1%).

The formula for the “Actual NTU Value After Dilution,” reproduced below, was obtained from the manual for the Hach 2100N turbidimeter (for formazin standard preparations).

$$NTU = \frac{1}{2} * \frac{( Vol\_of \_NTU \_Std )}{( Total\_Volume )} * NTU\_Std \_Value$$

where “NTU” is the “Actual NTU Value After Dilution”;

“Vol_of_NTU_Std” is the volume of NTU standard solution added;

“Total Volume” is the total volume in the cuvette; and

“NTU_Std_Value” is the NTU value of the standard solution.

Using that formula, applicant determined the increase in turbidity (in NTU) that should be calculated and displayed by the turbidimeter after the addition to 2.0 milliliters (2,000 microliters) of test fluid (pH 7 buffered aqueous solution) of 0.5 microliters of either the 1000 NTU standard solution, the 400 NTU standard solution, or the 200 NTU standard solution (formazin standard preparations).

For the 1000 NTU standard solution, the increase in NTU

$$= \frac{1}{2} * \frac{0.5 µL}{2 , 000 µL} + 1000 NTU$$

which equals 0.125 NTU per addition of 0.5 microliter to the 2.0 milliliters in the preferred cuvette. Similarly, for the 400 and 200 NTU standard solutions, the increases in NTU equal 0.05 NTU and 0.025 NTU, respectively, per addition of 0.5 microliter to the 2.0 milliliters in the cuvette.

Even though the calculated increase for each 0.5 microliter addition of the 1000 NTU standard solution is 0.125 NTU (and, therefore, that is the increase that should be displayed), the addition was found to cause an increase in the display reading on the turbidimeter of 0.003 NTU. Thus, the apparent increase in turbidity with the modified turbidimeter (which has the preferred 2.0 milliliter square lateral cross-section cuvette) for each 0.5 microliter addition of the 1000 NTU standard solution is only 0.003 NTU but the actual (calculated) increase in turbidity is 0.125 NTU. In other words, the modifications (including the use of the smaller, differently shaped preferred cuvette and the cuvette holder) desensitizes the Hach 2100N turbidimeter so that although the actual change in turbidity is 0.125 NTU, the turbidimeter calculates and displays a change of only 0.003 NTU. Thus, a 0.001 NTU change in the turbidity reading and signal sent to the turbidimeter LED display corresponds to an actual change of 0.042 NTU (i.e., 0.125 divided by 0.003). This information was used to further calibrate and characterize the preferred modified unit.

The following procedure was used to conduct a series of replicate runs for determining the standard deviation to use in calculating the “max” turbidity value, and the data obtained are plotted in FIG. 25. As discussed above in connection with the description of FIG. 4B, the standard deviation is used to determine the “max” allowable turbidity value of the system, above which maximum value (“max”) the cuvette must be cleaned (first with the dual solvent rinse and then if necessary with the more aggressive 3-cycle wash one, two, or three times). As discussed above in connection with FIG. 4C, the standard deviation is also used to establish the magnitude of increase in turbidity that indicates that a substance being added to a test fluid has come out of solution when the (preferred) concentration method is being used to determine kinetic solubility.

Using the preferred apparatus described above, the cuvette was filled with test fluid (a buffered solution of 7 pH) without any substance (i.e., test substance) being present, and a baseline turbidity reading was taken (“Baseline” value). A first aliquot of 0.5 microliters of 1000 NTU standard solution was added to the cuvette (“Addition Number” 1) and the turbidity was again measured. A second aliquot of the same volume of the same standard solution was added (“Addition Number” 2) and the turbidity was measured. Third, fourth, fifth, sixth, seventh, and eighth additions were made and the turbidity was measured after each addition. The resulting nine points (“Baseline” turbidity value before any addition and the turbidity value after each one of the eight additions) were plotted and connected. After this set of runs, the cuvette was emptied, cleaned, and the process of obtaining the nine data points was repeated using the 1000 NTU standard solution. Twelve such sets of runs were made, and the data points are plotted in FIG. 25 (some of the lines connecting the points are not visible because they are too close to other lines). For each “Addition Number” (i.e., for each of the nine X-axis values), a mean average turbidity value was calculated, and those average values are indicated by small squares. The standard deviation for each set of twelve turbidity values at each of the nine addition levels (Baseline and each of the eight additions of standard solution) was then calculated, and those nine standard deviations were averaged to determine a mean average standard deviation value of 0.001 NTU.

Use of the mean average standard deviation determined in this manner is reasonable because it is a mean average of standard deviations determined throughout the range of turbidity readings likely to be encountered in the actual kinetic solubility assay determinations. As can be seen in FIG. 25, the curves indicate that the increase in the turbidity with each addition of standard solution is very close to linear and that as the baseline value of the testable mixture increases, the ability to detect a change in turbidity resulting from a single addition of standard solution diminishes. Nevertheless, this experiment does demonstrate the repeatability of testing and determination of kinetic solubility using the apparatus and method of this invention: the coefficients of variation are low, ranging from 1.4% to 1.84%.

As noted above in connection with the discussion of FIG. 4C, the point at which a substance is indicated to have come out of solution (i.e., formed detectable particles in the testable mixture) is when the apparatus detects a change in turbidity of the testable mixture greater than three times the standard deviation of the system. It is preferred to use the standard deviation of the system value determined for additions of 1000 NTU standard solution (i.e., the 0.001 NTU value).

As a result of these experiments, the following values were established for the preferred apparatus based on testing using the 1000 NTU standard solution, the smallest volume addition that can be made by the precision pumps (i.e., 0.5 microliters), etc.

| Characteristic | Hach Display (NTU) | True (Adjusted) Value (NTU) |
|---|---|---|
| Resolution | 0.001 | 0.042 |
| Repeatability | ±0.003 | ±0.126* |

*Because of rounding, does not equal ±0.125

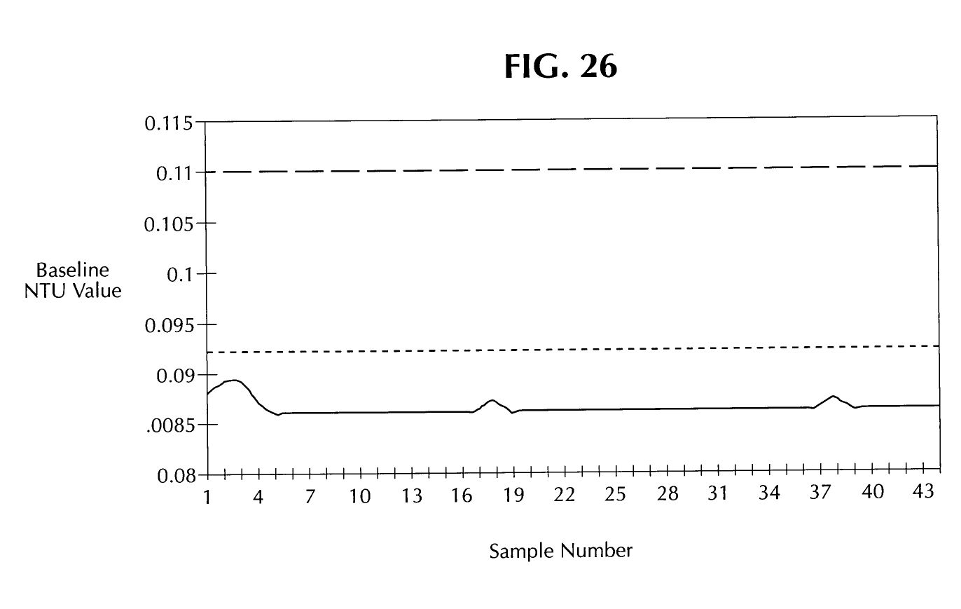

FIG. 26 shows baseline NTU values plotted against sample number, i.e., NTU values for the test fluid in the cuvette prior to test substance addition for the different samples. Also shown are two horizontal lines, the lower of which indicates an NTU value designated as “high” (approximately 0.092, as shown by the shorter broken lines) and the upper of which indicates an NTU value designated as the “max” (maximum) allowable (approximately 0.11, as shown by the longer broken lines).

As explained above, the “max” value corresponds to the degree of “dirtiness” a cuvette can have that no longer allows (with a sufficient degree of confidence) detection of a true increase in turbidity resulting from a single addition of test substance. Thus, the turbidity reported may be higher following addition of the test substance, but because of the high level of “dirtiness” of the cuvette, one no longer can have sufficient confidence that the turbidity has actually increased. Recall that a baseline value is determined at the start of each assay of an unknown, i.e., a turbidity value is determined after addition of the test fluid to the cuvette but before any of the unknown substance being tested has been added. When such a start-of-run baseline value equals or exceeds the “max” value, any observable increase in turbidity for the first and subsequent additions of test substance are likely to be masked by the random noise of the system (as explained below, the amount of increase in measured turbidity generally increases with each addition of test substance, i.e., until precipitation of a solid phase occurs). The random noise of a system with respect to a parameter (e.g., turbidity) may be considered to be within the range of plus and minus three standard deviations from the mean average of the parameter, because for a normal (bell-shaped) distribution, approximately 99.7% of all of the area under the curve lies within that range (from the mean minus three standard deviations to the mean plus three standard deviations). Thus, a three-standard deviation shift from the baseline represents a turbidity increase that is large enough (i.e., statistically significant) not to be considered an anomaly. In some instances, it may be preferable to use a larger baseline shift.

The “max” was determined in the following way. A set of tarry and gummy compounds were intermixed with 1000 NTU standard solution samples. As the set was being run, the cuvette became soiled and the baseline began to rise. Eventually, a single 0.5 microliter addition of the standard solution was no longer undetectable because the “noise floor” had risen so high, and that point was 0.11 NTU.

FIG. 26 presents data demonstrating the excellent cleanliness of the cuvette that can be maintained during the kinetic solubility assay using the preferred apparatus and process, including the preferred rinsing/cleaning steps and fluids. The test fluid was a pH 7.0 buffer and the test substances were randomly selected library compounds.

The “Baseline NTU Value” (Y-axis value) is the turbidity of the test fluid in the cuvette prior to the addition of any test substance to the test fluid. The “max” value determined for the system in the manner described above (i.e., the NTU value above which the cuvette is deemed too dirty to accurately assay the samples and report turbidity values in which one has sufficient confidence) and the “high” value are also plotted (the “high” value of 0.092 was set at about 84% of the “max” value (a value of 70% of “max” would have resulted in the cleaning cycle being run too often). The baseline turbidity for the first sample was about 0.088 NTU, increased to almost 0.09 NTU for the third sample, thereafter declined to just above 0.085 NTU and remained at that level until the eighteenth sample, when it rose slightly. The NTU value decreased to just above 0.085 NTU for the nineteenth sample and remained there until the thirty-eighth sample. It rose slightly for the thirty-ninth sample and then dropped and remained steady at just above 0.085 NTU until the end of the assay (the forty-fourth sample). At no point did the baseline turbidity reach even the “high” value.

A comparison was made between the earlier assay used internally by the assignee of the present application (described above) and the preferred kinetic solubility assay of this invention. Samples of 45 compounds (from the sample library) whose turbidities had previously been determined using the earlier assay were obtained. The compounds were diluted with DMSO to a concentration of 10 micrograms per milliliter, subjected to vortex mixing (to try to redissolve any solids), assayed for kinetic solubility using the earlier assay, and then assayed using the preferred apparatus and process of this invention. The assay of this invention was performed immediately after the earlier assay was run to minimize any intervening crystal formation.

There was excellent agreement between the results of the two assays, with a breakdown of the results as follows:

| | |
|---|---|
| No difference: | 82.2% (earlier assay and assay of this invention report |
| | the same kinetic solubility for a |
| | compound) |
| Less Sensitive: | 0% (assay of this invention reports higher solubility) |
| More Sensitive: | 17.8% (assay of this invention reports lower solubility) |

For eighty-nine percent (i.e., 40) of the pairs of kinetic solubilities (each pair consisting of the kinetic solubility from the earlier assay and the kinetic solubility from the assay of this invention), the two kinetic solubility values were within 10 micrograms/milliliter of each other, which, for the preferred assay of this invention using 10 micrograms of test compound per microliter of carrier DMSO, is equivalent to 2 additions each of 0.5 microliters. In other words, the kinetic solubility value determined by the preferred assay was either higher or lower or lower than the kinetic solubility value determined by the earlier assay by not more than 2 additions each of 0.5 microliters (as indicated above, the preferred maximum number of additions allowed is 40).

Another group of approximately 480 compounds that had previously been tested using the earlier assay were assayed using the preferred kinetic solubility assay of this invention. The compounds (in DMSO carrier fluid) had been left in their respective vials (after samples from them had been taken for the earlier assay) for periods ranging from several days to weeks. As a result, crystals formed in some of the vials. Vortexing was used (for 45 minutes) to try to redissolve the compounds in their carrier fluids. Nevertheless, there was a strong correlation between the results from the two assays, with an advantage to the assay of this invention in dealing with colored compounds because of the use of a LPF-650 filter in the assay of the present invention (when that filter was used with the older assay, some compounds that had been determined to be insoluble appeared to be soluble). A breakdown of the results is as follows:

| | |
|---|---|
| No difference: | 60.7% (earlier assay and assay of this invention report |
| | the same kinetic solubility for a |
| | compound) |
| Less Sensitive: | 13.5% (assay of this invention reports higher solubility) |
| More Sensitive: | 25.8% (assay of this invention reports lower solubility) |

Although approximately 39% pairs of results (each pair consisting of the result from the earlier assay and the result from the assay of this invention) did differ between the two assays, more often than not (i.e., 75% of the 39%), the difference was small (i.e., only a 5 micrograms/milliliter to 10 micrograms/milliliter difference in reported solubility).

In both assays (the earlier assay and the assay of this invention), the greatest degree of variability in results occurs in the middle region (between 5 micrograms per milliliter and 65 micrograms per milliliter). A result for a substance that has “very good” kinetic solubility (>65 micrograms per milliliter), in other words is highly soluble, or that has “poor” kinetic solubility (<5 micrograms per milliliter), in other words is essentially insoluble, has been found to be extremely repeatable.

A test batch of fourteen different compounds whose solubility was within the middle region of the assay range was run 3 times using the preferred apparatus and procedures. In other words, the solubilities for the fourteen substances used span the middle range of almost “poor” (about 5 micrograms per milliliter) to almost “very good” (about 65 micrograms per milliliter).

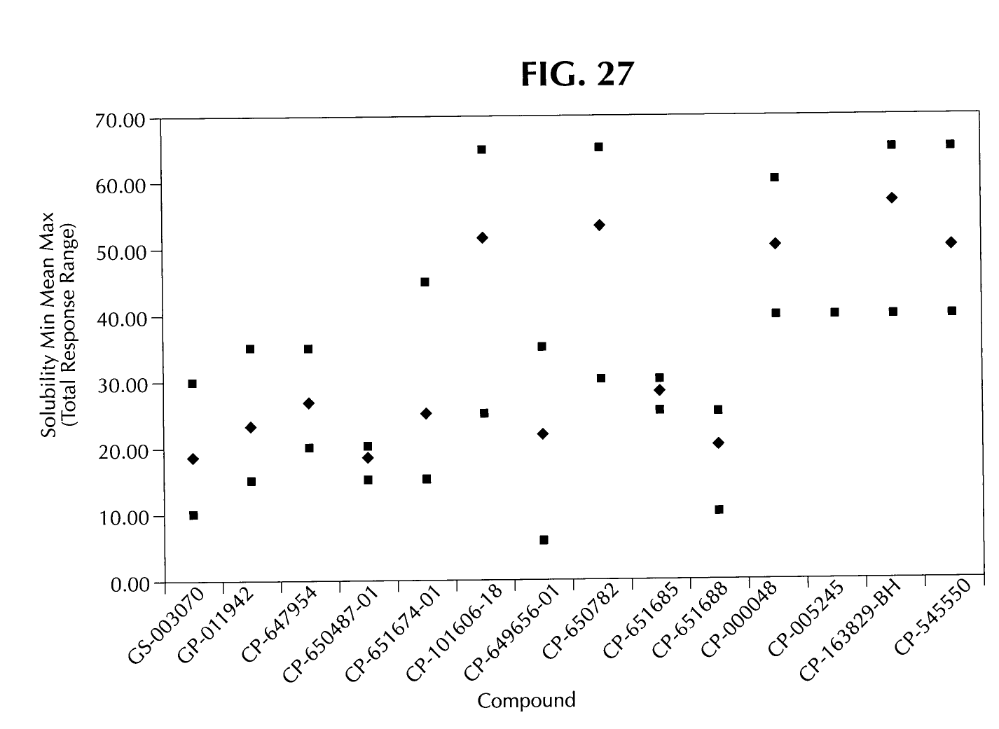

FIG. 27 presents kinetic solubility data obtained for those fourteen compounds (the compounds are indicated on the X-axis using designations for them employed internally by the assignee of the present application). The maximum value (indicated by the top small box for each compound), the average value (indicated by a diamond for each compound), and the minimum value (indicated by the lower small box for each compounds) are shown and a vertical line has been drawn to connect the three values for each compound. For compound CP-005245, the three values were the same (hence only a single box is shown for it).

The standard error of the kinetic solubilities ranges from 0 to 23 micrograms per milliliter, with a mean standard error of about 10 micrograms per milliliter. This indicates that the kinetic solubility value for a compound whose solubility is in this middle range (whether one of the fourteen compounds shown or a different compound) can be expected to have an error margin of about 10 micrograms per milliliter (and no greater than 25 micrograms per milliliter) if that same compound is assayed more than once.

As noted above, turbidimetric kinetic solubilities less than about 30 micrograms per milliliter usually exceed thermodynamic values by 2 to 4 times and that turbidimetric kinetic solubilities are comparable to thermodynamic solubilities for kinetic solubilities greater than about 50 micrograms per milliliter, with the following exceptions. If thermodynamic values are measured at a pH different from the pH used for determining kinetic solubility, there may be a difference in solubility values. For example, if thermodynamic values are measured at pH 6.5 and kinetic solubility assay values are determined at pH 7.0, there will likely be a difference in the solubility values for weak to moderate bases. For amorphous compounds or compounds forming very stable and insoluble hydrates, the kinetic solubility assay values will be markedly higher than the thermodynamic solubilities. For small micelle aggregates, the kinetic solubility assay will indicate that they are more soluble than is truly the case.

Submitted as an appendix to the specification of the present invention, is a source code listing for a preferred computer program that is resident in computer 46 and thereby operates the preferred kinetic solubility assay apparatus. The program is written in Visual Basic 5.0, Service Pack 3.0, in a Windows NT 4.0, Service Pack 3.0, environment, along with Component Works Virtual Instrument Tools (from National Instruments Co.), VisualLab Active X Controls (from I/O Tech Co.), Sheridan Developers Toolkit For Visual Basic (from Sheridan Developers Co.), and Port I/O DLL (from Scientific Software Tools Co.); however, any other suitable programming languages and environment could be used.

As should be apparent even without consideration of the source code listing, the inputs to the program include information received from the syringes, the RSP9000 Robotic Sample Processor, the Keithley MBB-32 32-channel I/O board, the custom turbidimeter interface board, and the rinse pump interface, and the outputs from the program include information sent to the syringes, the RSP9000 Robotic Sample Processor, the turbidimeter, the Keithley MEM-08 8-channel relay board, the Keithley MEM-32 32-channel relay board, valves, resets on the custom turbidimeter interface board, and the rinse pump interface. One skilled the art will know how to prepare a computer program for monitoring and controlling the particular automated kinetic solubility assay apparatus of this invention chosen for use for assessing the kinetic solubility.

In summary, the present invention provides apparatus that determines kinetic solubility rapidly, accurately, with good sensitivity, and with good reproducibility, that is automatic, requiring essentially no operator attention, that provides for increased safety (e.g., by reducing the risk of the operator's contacting the test substances and any carrier fluids, through use of septum-sealed containers and test chambers and use of the septum-piercing needle), that is reliable, that requires only very small amounts of test substances, that overcomes the problems associated with a small diameter round cuvette and with a square lateral cross-section cuvette, and that can screen large numbers of substances to determine their kinetic solubility with all of the above-noted advantages. The present invention also provides a cuvette, a needle, and cleaning solutions, each having its own features and advantages and each of which may be used in or with the automated kinetic solubility assay apparatus of this invention. Other features and advantages of the various aspects of the invention should be apparent to those skilled in the art.

The invention has been described in an illustrative manner and the terminology that has been used is intended to be in the nature of description rather than of limitation. Modifications and variations that can be made should be apparent in light of the teachings herein. It is, therefore, to be understood that within the scope of the appended claims, the invention can be practiced otherwise than as specifically described and that the claims are intended to cover all modifications and variations falling within the true spirit and scope of the invention.

I claim:

1. An automated kinetic solubility assay apparatus for assessing the kinetic solubility of one or more substances in one or more test fluids, the apparatus comprising:
(a) a cuvette for automatically receiving a first test fluid and a first substance, the cuvette having at least two spaced wall sections;
(b) test fluid addition means for automatically adding the first test fluid to the cuvette and substance addition means for automatically adding the first substance to the cuvette to cause initial contact of the first test fluid and first substance in the cuvette, thereby to produce a first testable mixture;
(c) turbidity measurement means for automatically measuring the turbidity of the first testable mixture in the cuvette using energy that is directed to pass through at least one of the two spaced wall sections of the cuvette, then the first testable mixture in the cuvette, and then through the other of the two spaced wall sections of the cuvette;
(d) kinetic solubility assessing means for automatically assessing the kinetic solubility of the first substance in the first test fluid from the turbidity of the first testable mixture in the cuvette;
(e) removal means for automatically removing the first testable mixture from the cuvette.

2. The apparatus of claim 1 further comprising cleaning means for automatically cleaning the cuvette to increase the transmittance of energy that can pass through at least its two spaced wall sections, and a cleaning activation means to automatically activate the cleaning means to automatically clean the cuvette to increase the transmittance of energy that can pass through at least its two spaced wall sections if the transmittance after the first testable mixture has been removed from the cuvette is below a predetermined value.

3. The apparatus of claim 1 wherein (i) the substance addition means comprises means for automatically repeatedly adding the first substance to the cuvette and/or the test fluid addition means comprises means for automatically repeatedly adding the first test fluid to the cuvette; (ii) the substance addition means further comprises means for halting the repeated addition of the first substance to the cuvette and/or the repeated addition of the first test fluid to the cuvette after the earlier of either of two conditions occurs: the number of additions of the first substance or the first test fluid to the cuvette exceeds a predetermined value or the turbidity of the first testable mixture in the cuvette is above or below a predetermined value, (iii) the turbidity measurement means comprises means for automatically measuring the turbidity of the first testable mixture in the cuvette after each addition of the first substance to the cuvette and/or after each addition of the first test fluid to the cuvette.

4. The apparatus of claim 3 wherein the kinetic solubility assessing means comprises means for automatically assessing the kinetic solubility after the addition of the first substance and/or of the first test fluid to the cuvette has been halted.

5. The apparatus of claim 1 further comprising means to automatically rinse the cuvette after the first testable mixture has been removed from the cuvette by the removal means.

6. The apparatus of claim 1 further comprising means to cause:
(a) the test fluid addition means to automatically add a second test fluid to the cuvette and the substance addition means to automatically add a second substance to the cuvette to cause initial contact of the second test fluid and second substance in the cuvette, thereby to produce a second testable mixture, the addition of the second test fluid and second substance to the cuvette occurring after the cuvette has been rinsed;
(b) the turbidity measurement means to automatically measure the turbidity of the second testable mixture in the cuvette using energy that is directed to pass through at least one of the two spaced wall sections of the cuvette, then the second testable mixture in the cuvette, and then through the other of the two spaced wall sections of the cuvette;
(c) the kinetic solubility assessing means to automatically assess the kinetic solubility of the second substance in the second test fluid from the turbidity of the second testable mixture in the cuvette; and
(d) the removal means to automatically remove the second testable mixture from the cuvette.

7. The apparatus of claim 6 further comprising means for determining the transmittance of energy passing through at least the two spaced wall sections of the cuvette in the absence of the first substance, in the absence of the second substance, and after the first testable mixture has been removed from the cuvette by the removal means and before the second testable mixture is present in the cuvette.

8. The apparatus of claim 7 further comprising cleaning means for automatically cleaning the cuvette to increase the transmittance of energy that can pass through at least its two spaced wall sections.

9. The apparatus of claim 8 further comprising cleaning activation means to automatically activate the cleaning means to automatically clean the cuvette to increase the transmittance of energy that can pass through at least its two spaced wall sections if the transmittance in the absence of the first substance and second substance and after the first testable mixture has been removed from the cuvette by the removal means and before the second testable mixture is present in the cuvette is below a predetermined value.

10. The apparatus of any of claims 1 to 9 wherein the cuvette has a volume of from 0.3 to 5 milliliters and further comprises a first planar surface and a second planar surface parallel to the first planar surface and one of the two spaced wall sections of the cuvette is a portion of the first parallel planar surface and the other spaced wall section of the cuvette is a portion of the second parallel planar surface.

11. The apparatus of any of claims 1 to 9 wherein the first substance is in a container having a pierceable septum, the cuvette comprises a pierceable septum, and the apparatus further comprises needle manipulation means and a septum-piercing needle having a passageway, the needle and needle manipulation means being for (i) piercing the septum of the container with the needle, (ii) withdrawing the first substance from the container after the needle pierces the septum of the container and holding the withdrawn first substance in at least the passageway of the needle, (iii) withdrawing the needle from the septum of the container and piercing the septum of the cuvette with the needle, and (iv) discharging the withdrawn first substance from at least the passageway of the needle into the cuvette after the needle pierces the septum of the cuvette.

12. The apparatus of any of claims 1 to 9 comprising a plurality of cuvettes, each cuvette being operatively associated with test fluid addition means, substance addition means, turbidity measurement means, and removal means.

13. A septum-piercing needle having a straight upper portion, a curved lower portion, a longitudinal axis, a piercing end at the end of the curved lower portion, and a non-piercing end in the straight upper portion; the needle comprising a rigid exoskeleton lined with a corrosion-resistant cannula having a central elongate passageway running from the piercing end of the needle to the non-piercing end of the needle, the passageway of the cannula has an average diameter and a length and the average diameter is from 100 to 300 microns and the length is from 10 to 150 millimeters, the passageway of the cannula further being adapted to hold fluid and terminating at the piercing end of the needle in an opening, the piercing end of the needle being adapted for piercing the pierceable septum of a container holding fluid to allow fluid to be withdrawn from the container and to flow through the opening of the passageway at the piercing end of the cannula into the passageway of the cannula, the plane of the opening of the passageway at the piercing end of the needle being substantially parallel to the longitudinal axis of the straight upper portion of the needle.

14. A cuvette in which a fluid sample may be placed for testing, the cuvette comprising:
(a) a bottom, a top, and a wall therebetween and connected to both, the bottom, the top, and the wall together defining an enclosed volumetric space for receiving fluid;
(b) a pierceable septum forming part of the top to allow fluid to be injected through the septum into the volumetric space within the cuvette;
(c) means to remove fluid from the volumetric space within the cuvette; and
(d) a vent fluidly communicating between the volumetric space and the region outside of the cuvette through which (i) gas in the volumetric space in the cuvette can flow to the region outside the cuvette as fluid is injected into the volumetric space in the cuvette through the pierceable septum and (ii) gas in the region outside of the cuvette can flow into the volumetric space in the cuvette when fluid is removed from the volumetric space in the cuvette.

15. A cleaning agent comprising a mixture of ethylenediaminetetraacetic acid from 0.001% w to 50% w and glass cleaner having water, ammonium hydroxide, 2-propanol, 2-butoxy ethanol, and anionic surfactant.
